(19) United States
(12) Patent Application Publication
(10) Pub. No.: US 2024/0000882 A1
(43) Pub. Date: Jan. 4, 2024

(54) COMPOSITIONS AND METHODS FOR BODY CONTOURING AND SURGICAL PROCEDURES

(71) Applicant: ALASTIN SKINCARE, INC., Carlsbad, CA (US)

(72) Inventors: Alan David WIDGEROW, Carlsbad, CA (US); John A. GARRUTO, Carlsbad, CA (US)

(73) Assignee: ALASTIN SKINCARE, INC., Carlsbad, CA (US)

(21) Appl. No.: 18/223,305

(22) Filed: Jul. 18, 2023

Related U.S. Application Data
(63) Continuation of application No. PCT/US22/12796, filed on Jan. 18, 2022.
(60) Provisional application No. 63/139,088, filed on Jan. 19, 2021.
(60) Provisional application No. 63/225,857, filed on Jul. 26, 2021.
(60) Provisional application No. 63/289,042, filed on Dec. 13, 2021.

(51) Int. Cl. A61K 38/08; A61K 38/06; A61K 38/07; A61K 38/17; A61K 9/00; A61P 29/00; A61K 45/06

(52) U.S. Cl. CPC A61K 38/08; A61K 38/06; A61K 38/07; A61K 45/06; A61K 9/0014; A61P 29/00; A61K 38/1741

(57) ABSTRACT

Disclosed herein are topical compositions and methods of use for improving or ameliorating effects associated with body contouring or surgical procedures. Compositions as described herein comprise one or more peptides.

### CROSS-REFERENCE

This application is a continuation of International Application No. PCT/US2022/012796 filed Jan. 18, 2022, which application claims benefit of U.S. Provisional Application No. 63/139,088 filed Jan. 19, 2021, U.S. Provisional Application 63/225,857 filed Jul. 26, 2021, and U.S. Provisional Application No. 63/289,042 filed Dec. 13, 2021, all of which are herein incorporated by reference in their entireties.

### BACKGROUND

Traditional and conventional skincare treatments for alleviating negative side effects resulting from surgeries and other body contouring procedures have primarily relied upon a variety of common over the counter remedies. The need for treatments effective at ameliorating the effects of body contouring or surgical procedures is rapidly growing.

### BRIEF SUMMARY

Described herein are topical compositions and methods of use for body contouring or surgical procedures. In some embodiments, the topical compositions are used in preparation for a body contouring or surgical procedure. In some embodiments, the topical compositions are used after a body contouring or surgical procedure. In some embodiments, the topical compositions are used both for preparation for a body contouring or surgical procedure and after a body contouring or surgical procedure.

An aspect described herein are topical compositions comprising: a tripeptide-1; a tetrapeptide-2; a hexapeptide-12; a hexapeptide-11; and a glycoprotein. In one feature, the tripeptide-1 is present in a range of about 0.05% by weight (wt. %) to about 5.00% wt. %. In one feature, the tripeptide-1 is present at about 3.00 wt. %. In one feature, the tripeptide-1 comprises palmitoyl tripeptide-1, myristoyl tripeptide-1, or a combination thereof. In one feature, the hexapeptide-12 comprises palmitoyl hexapeptide-12, myristoyl hexapeptide-12, or a combination thereof. In one feature, the hexapeptide-12 is present in a range of about 0.05 wt. % to about 5 wt. %. In one feature, the hexapeptide-12 is present at about 3.00 wt. %. In one feature, the hexapeptide-11 is present in a range of about 0.002 wt. % to about 0.5 wt. %. In one feature, the hexapeptide-11 is present at about 0.01 wt. %. In one feature, the glycoprotein is a transferrin. In one feature, the glycoprotein is a lactoferrin. In one feature, the lactoferrin is present in a range of about 0.01 wt. % to about 0.3 wt. %. In one feature, the lactoferrin is present at no more than about 0.01 wt. %. In one feature, the lactoferrin is present at about 0.05 wt. %. In one feature, the composition further comprises phosphatidylserine. In one feature, the phosphatidylserine is present at no more than about 0.002 wt. %. In one feature, the phosphatidylserine is present in a range of about 0.002 wt. % to about 0.5 wt. %. In one feature, the phosphatidylserine is present at no more than about 5.0 wt. %. In one feature, the phosphatidylserine is present at about 0.1 wt. %. In one feature, the composition further comprises oleuropein. In one feature, the oleuropein is present at no more than about 0.01 wt. %. In one feature, the oleuropein is present in a range between about 0.01 wt. % and about 0.30 wt. %. In one feature, the oleuropein is present at about 0.05 wt. %. In one feature, the composition further comprises Tremella fuciformis extract. In one feature, the Tremella fuciformis extract is present at no more than about 0.10 wt. %. In one feature, the Tremella fuciformis extract is present in a range of about 0.10 wt. % to about 3.00 wt. %. In one feature, the Tremella fuciformis extract is present at about 0.50 wt. %. In one feature, the composition further comprises Peucedanum graveolens extract. In one feature, the Peucedanum graveolens extract is present at no more than about 0.10 wt. %. In one feature, the Peucedanum graveolens extract is present in a range of about 0.10 wt. % to about 3.00 wt. %. In one feature, the Peucedanum graveolens extract is present at about 0.50 wt. %. In one feature, the composition further comprises hydroxymethoxyphenyl decanone. In one feature, the hydroxymethoxyphenyl decanone is present at no more than about 0.10 wt. %. In one feature, the hydroxymethoxyphenyl decanone is present in a range of about 0.10 wt. % to about 3.00 wt. %. In one feature, the hydroxymethoxyphenyl decanone is present at about 0.50 wt. %. In one feature, the composition further comprises Dunaliella salina extract. In one feature, the Dunaliella salina extract is present at no more than about 0.10 wt. %. In one feature, the Dunaliella salina extract is present in a range of about 0.10 wt. % to about 3.00 wt. %. In one feature, the Dunaliella salina extract is present at about 0.50 wt. %. In one feature, the composition further comprises Ledum palustre. In one feature, the Ledum palustre is present at no more than about 0.10 wt. %. In one feature, the Ledum palustre is present in a range of about 0.10 wt. % to about 3.00 wt. %. In one feature, the Ledum palustre is present at about 0.50 wt. %. In one feature, the composition further comprises xylitylglucoside. In one feature, the xylitylglucoside is present at no more than about 0.2 wt. %. In one feature, the xylitylglucoside is present in a range of about 0.2 wt. % to about 5.00 wt. %. In one feature, the xylitylglucoside is present at about 1.00 wt. %. In one feature, the composition further comprises anhydroxylitol. In one feature, the anhydroxylitol is present at no more than about 0.2 wt. %. In one feature, the anhydroxylitol is present in a range of about 0.2 wt. % to about 5.00 wt. %. In one feature, the anhydroxylitol is present at about 1.00 wt. %. In one feature, the composition further comprises xylitol. In one feature, the xylitol is present at no more than about 0.2 wt. %. In one feature, the xylitol is present in a range of about 0.2 wt. % to about 5.00 wt. %. In one feature, the xylitol is present at about 1.00 wt. %. In one feature, the composition further comprises xylitylglucoside, anhydroxylitol, and xylitol. In one feature, the xylitylglucoside, anhydroxylitol, and xylitol are present at no more than about 0.2 wt. %. In one feature, the xylitylglucoside, anhydroxylitol, and xylitol are present in a range of about 0.2 wt. % to about 5.00 wt. %. In one feature, the xylitylglucoside, anhydroxylitol, and xylitol are present at about 1.00 wt. %. In one feature, the composition further comprises Centella asiatica extract. In one feature, the Centella asiatica extract is present at no more than about 0.2 wt. %. In one feature, the Centella asiatica extract is present in a range of about 0.2 wt. % to about 5.00 wt. %. In one feature, the Centella asiatica extract is present at about 1.00 wt. %. In one feature, the composition further comprises naringenin. In one feature, the naringenin is present at no more than about 0.4 wt. %. In one feature, the naringenin is present in a range of about 0.4 wt. % to about 10.00 wt. %. In one feature, the naringenin is present at about 2.00 wt. %. In one feature, the composition further comprises Arnica montana extract. In one feature, the Arnica montana extract is present at no more than about 0.10 wt. %. In one feature, the Arnica montana extract is present in a range of about 0.10 wt. % to about 3.00 wt. %. In one feature, the Arnica montana extract is present at about 0.50 wt. %. In one feature, the composition further comprises tetrandrine. In one feature, the tetrandrine is present at no more than about 0.0002 wt. %. In one feature, the tetrandrine is present in a range of about 0.0002 wt. % to about 0.005 wt. %. In one feature, the tetrandrine is present at about 0.001 wt. %. In one feature, the topical composition is aqueous. A method for improving or preventing effects of a body contouring or surgical procedure comprising administering the topical compositions described herein.

### BRIEF DESCRIPTION OF THE DRAWINGS

FIG. 1 illustrates a graph of TGF-β3 expression following treatment with tripeptide-1 (Tri), hexapeptide-12 (Hex12), and tripeptide-1 and hexapeptide-12 (TriHex).

FIGS. 2A-2B illustrate images from a subject from a split body trial with liposuction lateral aspects of both breasts as part of body contour surgery. Comparison of investigator assessed induration in pre-conditioned right breast (FIG. 2A) compared with non-preconditioned breast (FIG. 2B). Areas of induration, edema believed to correspond with inflammasomes prior to resolution.

FIG. 3 illustrates that the use of the composition with TriHex significantly improved ecchymosis at post-operative day (POD) 10-14 as compared to the bland moisturizer as assessed by mean blinded investigator assessment scores, where p<0.05. The mean blinded investigator assessment scores were similar for subcutaneous fibrous banding, skin discoloration, edema, VAS, and induration at POD 10-14.

FIG. 4 shows the mean blinded investigator assessment scores at post-operative day (POD) 21-25. The use of the composition with TriHex resulted in significantly improved subcutaneous fibrous banding and skin discoloration at POD 21-25 (p<0.05).

FIG. 5 shows the mean blinded investigator assessment scores at POD 28-30. The use of the composition with TriHex resulted in significantly improved subcutaneous fibrous banding and edema at POD 28-30 (p<0.05).

FIG. 6 shows the mean blinded investigator assessment scores at POD 42-50. The use of the composition with TriHex resulted in significantly improved subcutaneous fibrous banding, skin discoloration, and edema at POD 42-50 (p<0.05).

FIG. 7 shows that a statistically significant proportion of subjects reported less bruising and discoloration on the side treated with the composition with TriHex (48.5% of subjects) as compared to the side treated with bland moisturizer (30.3% of subjects), with a p<0.0001 at all follow-up time points. 21.2% of the subjects reported no difference. This may indicate accelerated healing on the side treated with the composition with TriHex.

FIG. 8 shows that the mean blinded subject scores of the group treated with the composition with TriHex demonstrated a significant improvement over bland moisturizer in subcutaneous fibrous banding (1 for bland moisturizer vs 0.8 for the composition) and skin discoloration (1.3 vs 1.1) at POD 21-25.

FIGS. 9A-C show images of the hips and thighs of a 62 years old subject after a lateral thigh liposuction who received two weeks of pre-treatment and 12 weeks of post-treatment with the composition with TriHex vs. bland moisturizer at POD 1-3 (FIG. 9A), POD 5-7 (FIG. 9B), and POD 10-14 (FIG. 9C). The images demonstrate reduced skin discoloration, ecchymosis, edema with the application of the composition with TriHex before and after the procedure as compared to the control group with a bland moisturizer.

FIGS. 10A-D show images of the abdomen of a 38 year old subject after an abdominoplasty and liposuction who received two weeks of pre-treatment and 12 weeks of post-treatment with the composition with TriHex vs. bland moisturizer at POD 21-25 (FIGS. 10A, B) and POD 42-50 (FIGS. 10C, D). The images demonstrate reduced fibrous banding in the areas indicated by a circle in FIGS. 10A and 10C the application of the composition with TriHex before and after the procedure as compared to the control group with a bland moisturizer in FIGS. 10B and 10D.

FIGS. 11A-D show images of the breast of a subject after a secondary breast augmentation who received two weeks of pre-treatment and 12 weeks of post-treatment with the composition with TriHex vs. bland moisturizer at POD 21-25 (FIGS. 11B, A) and POD 42-50 (FIGS. 10D, C). The images demonstrate reduced swelling in the areas indicated by a circle in FIGS. 11B and 11D the application of the composition before and after the procedure as compared to the control group with a bland moisturizer in FIGS. 11A and 11C.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

### Definitions

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an”, and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

### Compositions

Compositions described herein, in some embodiments, improve the effects following a body contouring or surgical procedure. In some embodiments, the compositions as described herein are used in preparation for a body contouring or surgical procedure, for example, as a pre-conditioning. In some embodiments, the compositions as described herein are used both for preparation for a body contouring or surgical procedure and after a body contouring or surgical procedure. In some embodiments, compositions as described herein are used to improve or prevent effects associated with body contouring or surgical procedure including, but not limited to, bruising, swelling, fat dissolution, antimicrobial effects, skin tightening, and scarring. In some embodiments, compositions as described herein are used to improve healing. In some embodiments, compositions as described herein improve healing by modulating adipocytolysis. In some embodiments, compositions as described herein improve healing by stimulating macrophage function. Compositions as described herein, in some embodiments, comprise lactoferrin, tripeptide-1, hexapeptide-12, hexapeptide-11, phosphatidylserine, tetrapeptide-2, Peucedanum graveolens extract, hydroxymethoxyphenyl decanone, Dunaliella salina extract, Tremella extract, betaine, Ledum palustre extract, xylitylglucoside, anhydroxylitol, xylitol, oleuropein, Centella asiatica extract, naringenin, Arnica montana flower extract, tetrandrine, or combinations thereof. In some embodiments, the compositions described herein comprise in some embodiments, comprise lactoferrin, tripeptide-1, hexapeptide-12, hexapeptide-11, phosphatidylserine, tetrapeptide-2, Peucedanum graveolens extract, hydroxymethoxyphenyl decanone, Dunaliella salina extract, Tremella extract, betaine, Ledum palustre extract, xylitylglucoside, anhydroxylitol, xylitol, oleuropein, Centella asiatica extract, naringenin, Arnica montana flower extract, and tetrandrine.

Peptides

Compositions as described herein comprise one or more peptides. In some embodiments, the one or more peptides comprises tripeptide-1. In some embodiments, the one or more peptides comprises hexapeptide-12. In some embodiments, the one or more peptides comprises hexapeptide-11. In some embodiments, the one or more peptides comprises tetrapeptide-2. In some embodiments, the one or more peptides comprises hexapeptide-11, tripeptide-1, and hexapeptide-12. In some embodiments, the one or more peptides comprises hexapeptide-11, tripeptide-1, hexapeptide-12, tetrapeptide-2, or combinations thereof. In some embodiments, the one or more peptides comprises hexapeptide-11, tripeptide-1, hexapeptide-12, and tetrapeptide-2.

Compositions as described herein comprise a varying concentration of peptide. In some instances, a peptide is present at about 50 ppm or less to 1000, 5000, 10000, 50000, 100000, 500000 ppm or more, e.g., 100 ppm of the peptide. In some instances, a peptide is present at about 1, 2, 3, 4, 5, 6, 7, 8, 9, 10, 15, 20, 25, 50, 75, 100, 200, 300, 400, 500, 600, 700, 800, 900, 1000, or more than 1000 ppm. In some instances, a peptide is present in a range of about 1 to about 100, about 1 to about 50, about 1 to about 40, about 1 to about 30, about 1 to about 20, about 1 to about 10, about 5 to about 90, about 10 to about 80, about 20 to about 60, or about 30 to about 50 ppm. In some instances, a peptide is present at about 1, 2, 3, 4, 5, 6, 7, 8, 9, 10, 15, 20, 25, 50, 75, 100, 200, 300, 400, 500, 600, 700, 800, 900, 1000, or more than 1000 microgram per milliliter (μg/ml). In some instances, a peptide is present in a range of about 1 to about 100, about 1 to about 50, about 1 to about 40, about 1 to about 30, about 1 to about 20, about 1 to about 10, about 5 to about 90, about 10 to about 80, about 20 to about 60, or about 30 to about 50 microgram per milliliter. In some instances, a peptide is present from about 0.0001 wt. % to about 0.01 wt. %, 0.01 wt. % to about 10 wt. %, about 0.01 wt. % to about 0.02 wt. %, about 0.01 wt. % to about 0.03 wt. %, about 0.01 wt. % to about 0.04 wt. %, about 0.01 wt. % to about 0.05 wt. %, about 0.01 wt. % to about 0.1 wt. %, about 1 wt. % to about 5 wt. %, or about 1 wt. % to about 10% wt. %.

Compositions as described herein, in some embodiments, comprise one or more peptides. In some instances, a peptide of the one or more peptides is present at about 50 ppm or less to 1000, 5000, 10000, 50000, 100000, 500000 ppm or more, e.g., 100 ppm of the peptide, or any other suitable amount. In some instances, a peptide of the one or more peptides is present at about 1, 2, 3, 4, 5, 6, 7, 8, 9, 10, 15, 20, 25, 50, 75, 100, 200, 300, 400, 500, 600, 700, 800, 900, 1000, or more than 1000 ppm. In some instances, a peptide of the one or more peptides is present in a range of about 1 to about 100, about 1 to about 50, about 1 to about 40, about 1 to about 30, about 1 to about 20, about 1 to about 10, about 5 to about 90, about 10 to about 80, about 20 to about 60, or about 30 to about 50 ppm. In some instances, a peptide of the one or more peptides is present at about 1, 2, 3, 4, 5, 6, 7, 8, 9, 10, 15, 20, 25, 50, 75, 100, 200, 300, 400, 500, 600, 700, 800, 900, 1000, or more than 1000 microgram per milliliter (μg/ml). In some instances, a peptide of the one or more peptides is present in a range of about 1 to about 100, about 1 to about 50, about 1 to about 40, about 1 to about 30, about 1 to about 20, about 1 to about 10, about 5 to about 90, about 10 to about 80, about 20 to about 60, or about 30 to about 50 microgram per milliliter. In some instances, a peptide of the one or more peptides is present from about 0.01 wt. % to about 10 wt. %, about 0.01 wt. % to about 0.02 wt. %, about 0.01 wt. % to about 0.03 wt. %, about 0.01 wt. % to about 0.04 wt. %, about 0.01 wt. % to about 0.05 wt. %, about 0.01 wt. % to about 0.1 wt. %, about 1 wt. % to about 5 wt. %, or about 1 wt. % to about 10 wt. %. In some embodiments, a peptide of the one or more peptides is provided at least or about 0.00001 wt. %, 0.0003 wt. %, 0.0005 wt. %, 0.001 wt. %, 0.001 wt. %, 0.005 wt. %, 0.0055 wt. %, 0.01 wt. %, 0.02 wt. %, 0.05 wt. %, 0.10 wt. %, 0.25 wt. %, 0.50 wt. %, 0.75 wt. %, 1.0 wt. %, 1.5 wt. %, 2.0 wt. %, 2.5 wt. %, 3.0 wt. %, 3.5 wt. %, 4.0 wt. %, 4.5 wt. %, 5.0 wt. %, 5.5 wt. %, 6.0 wt. %, 6.5 wt. %, 7.0 wt. %, 8 wt. %, 9 wt. %, 10 wt. %, or more than 10 wt. %. In some embodiments, a peptide of the one or more peptides is provided in a range of about 0.25 wt. % to about 10 wt. %, about 0.5 wt. % to about 8 wt. %, about 0.75 wt. % to about 6 wt. %, or about 1 wt. % to about 4 wt. %. In some embodiments, each peptide of the one or more peptides is provided in a range of about 0.001 wt. % to about 6 wt. %, about 0.002 wt. % to about 4 wt. %, about 0.01 wt. % to about 3 wt. %, or about 0.02 wt. % to about 2 wt. %. In some embodiments, the peptide is tripeptide-1, hexapeptide-12, tetrapeptide-2, hexapeptide-11, or combinations thereof.

In compositions, the tripeptide is typically present in an amount of from about 50 ppm or less to about 100, 200, 300, 400, or 500 ppm or more, e.g., 50 ppm to 150 ppm. In compositions, the hexapeptide is typically present in an amount of from about 50 ppm or less to about 100, 200, 300, 400, or 500 ppm or more, e.g., 50 ppm to 150 ppm. In compositions, the tetrapeptide is present at 1-10 ppm. In one feature, a tetrapeptide is tetrapeptide-2. In compositions, the tetrapeptide-2 comprises acetyl tetrapeptide-2.

In some embodiments, the tripeptide-1 is provided at least or about 0.00001 wt. %, 0.0003 wt. %, 0.0005 wt. %, 0.001 wt. %, 0.001 wt. %, 0.00 wt. 5%, 0.0055 wt. %, 0.05 wt. %, 0.10 wt. %, 0.25 wt. %, 0.50 wt. %, 0.75 wt. %, 1.0 wt. %, 1.5 wt. %, 2.0 wt. %, 2.5 wt. %, 3.0 wt. %, 3.5 wt. %, 4.0 wt. %, 4.5 wt. %, 5.0 wt. %, 5.5 wt. %, 6.0 wt. %, 6.5 wt. %, 7.0 wt. %, 8 wt. %, 9 wt. %, 10 wt. %, or more than 10 wt. %. In some embodiments, the tripeptide-1 is provided in a range of about 0.6 wt. % to about 15 wt. %, about 0.25 wt. % to about 10 wt. %, about 0.5 wt. % to about 8 wt. %, about 0.75 wt. % to about 6 wt. %, or about 1 wt. % to about 4 wt. %. In some embodiments, the tripeptide-1 is provided at about 3 wt. %. In some embodiments, the tripeptide-1 is provided at least or about 0.25, 0.5, 0.75, 1, 1.5, 2, 2.5, 3, 3.5, 4, 5, 6, 7, 8, 9, 10, 15, 20, 25, or more than 25 ppm. In some embodiments, the tripeptide-1 is provided in a range of about 0.25 to about 10, about 0.5 to about 8, about 1 to about 6, or about 2 to about 4 ppm. In some embodiments, the tripeptide-1 is provided in a range of about 1 to about 10 ppm. In some embodiments, the tripeptide-1 is provided at least or about 0.25, 0.5, 0.75, 1, 1.5, 2, 2.5, 3, 3.5, 4, 5, 6, 7, 8, 9, 10, 15, 20, 25, or more than 25 microgram per milliliter (μg/ml). In some embodiments, the tripeptide-1 is provided in a range of about 0.25 to about 10, about 0.5 to about 8, about 1 to about 6, or about 2 to about 4 microgram per milliliter.

In some embodiments, the tetrapeptide-2 is provided at least or about 0.05 wt. %, 0.10 wt. %, 0.25 wt. %, 0.50 wt. %, 0.75 wt. %, 1.0 wt. %, 1.5 wt. %, 2.0 wt. %, 2.5 wt. %, 3.0 wt. %, 3.5 wt. %, 4.0 wt. %, 4.5 wt. %, 5.0 wt. %, 5.5 wt. %, 6.0 wt. %, 6.5 wt. %, 7.0 wt. %, 8 wt. %, 9 wt. %, 10 wt. %, or more than 10 wt. %. In some embodiments, the tetrapeptide-2 is provided in a range of about 0.2 wt. % to about 5 wt. %, about 0.25 wt. % to about 10 wt. %, about 0.5 wt. % to about 8 wt. %, about 0.75 wt. % to about 6 wt. %, or about 1 wt. % to about 4 wt. %. In some embodiments, the tetrapeptide-2 is provided at or about 1.00 wt. %. In some embodiments, the tetrapeptide-2 is provided at least or about 0.25, 0.5, 0.75, 1, 1.5, 2, 2.5, 3, 3.5, 4, 5, 6, 7, 8, 9, 10, 15, 20, 25, or more than 25 ppm. In some embodiments, the tetrapeptide-2 is provided in a range of about 1 to about 10 ppm. In some embodiments, the tetrapeptide-2 is provided in a range of about 0.25 to about 10, about 0.5 to about 8, about 1 to about 6, or about 2 to about 4 ppm. In some embodiments, the tetrapeptide-2 is provided at least or about 0.25, 0.5, 0.75, 1, 1.5, 2, 2.5, 3, 3.5, 4, 5, 6, 7, 8, 9, 10, 15, 20, 25, or more than 25 microgram per milliliter (μg/ml). In some embodiments, the tetrapeptide-2 is provided in a range of about 0.25 to about 10, about 0.5 to about 8, about 1 to about 6, or about 2 to about 4 microgram per milliliter.

In some embodiments, the hexapeptide-12 is provided at least or about 0.00001 wt. %, 0.0003 wt. %, 0.0005 wt. %, 0.001 wt. %, 0.001 wt. %, 0.005 wt. %, 0.0055 wt. %, 0.05 wt. %, 0.10 wt. %, 0.25 wt. %, 0.50 wt. %, 0.75 wt. %, 1.0 wt. %, 1.5 wt. %, 2.0 wt. %, 2.5 wt. %, 3.0 wt. %, 3.5 wt. %, 4.0 wt. %, 4.5 wt. %, 5.0 wt. %, 5.5 wt. %, 6.0 wt. %, 6.5 wt. %, 7.0 wt. %, 8 wt. %, 9 wt. %, 10 wt. %, or more than 10% wt. %. In some embodiments, the hexapeptide-12 is provided in a range of about 0.6 wt. % to about 15 wt. %, about 0.25 wt. % to about 10 wt. %, about 0.5 wt. % to about 8 wt. %, about 0.75 wt. % to about 6 wt. %, or about 1 wt. % to about 4 wt. %. In some embodiments, the hexapeptide-12 is provided at about 3 wt. %. In some embodiments, the hexapeptide-12 is provided at least or about 0.25, 0.5, 0.75, 1, 1.5, 2, 2.5, 3, 3.5, 4, 5, 6, 7, 8, 9, 10, 15, 20, 25, or more than 25 ppm. In some embodiments, the hexapeptide-12 is provided in a range of about 1 to about 10 ppm. In some embodiments, the hexapeptide-12 is provided in a range of about 0.25 to about 10, about 0.5 to about 8, about 1 to about 6, or about 2 to about 4 ppm. In some embodiments, the hexapeptide-12 is provided at least or about 0.25, 0.5, 0.75, 1, 1.5, 2, 2.5, 3, 3.5, 4, 5, 6, 7, 8, 9, 10, 15, 20, 25, or more than 25 microgram per milliliter (μg/ml). In some embodiments, the hexapeptide-12 is provided in a range of about 0.25 to about 10, about 0.5 to about 8, about 1 to about 6, or about 2 to about 4 microgram per milliliter.

In some embodiments, the hexapeptide-11 is provided at least or about 0.00001 wt. %, 0.0003 wt. %, 0.0005 wt. %, 0.001 wt. %, 0.001 wt. %, 0.005 wt. %, 0.0055 wt. %, 0.01 wt. %, 0.02 wt. %, 0.05 wt. %, 0.10 wt. %, 0.25 wt. %, 0.50 wt. %, 0.75 wt. %, 1.0 wt. %, 1.5 wt. %, 2.0 wt. %, 2.5 wt. %, 3.0 wt. %, 3.5 wt. %, 4.0 wt. %, 4.5 wt. %, 5.0 wt. %, 5.5 wt. %, 6.0 wt. %, 6.5 wt. %, 7.0 wt. %, 8 wt. %, 9 wt. %, 10 wt. %, 20 wt. %, 30 wt. %, 40 wt. %, 50 wt. %, 60 wt. %, 70 wt. %, 80 wt. %, 90 wt. % or more than 90 wt. %. In some embodiments, the hexapeptide-11 is provided in a range of about 0.25 wt. % to about 10 wt. %, about 0.5 wt. % to about 8 wt. %, about 0.75 wt. % to about 6 wt. %, or about 1 wt. % to about 4 wt. %. In some embodiments, the hexapeptide-11 is provided in a range of about 0.002 wt. % to about 0.05 wt. %, about 0.001 wt. % to about 6 wt. %, about 0.002 wt. % to about 4 wt. %, about 0.01 wt. % to about 3 wt. %, or about 0.02 wt. % to about 2 wt. %. In some embodiments, the hexapeptide-11 is provided in a range of about 0.005 wt. % to about 0.02 wt. %. In some embodiments, the hexapeptide-11 is provided at about 0.01 wt. %. In some embodiments, the hexapeptide-11 is provided at least or about 0.1 ppm, 3 ppm, 5 ppm, 10 ppm, 50 ppm, 55 ppm, 500 ppm, 1,000 ppm, 2,500 ppm, 5,000 ppm, or more than 5,000 ppm. In some embodiments, the hexapeptide-11 is provided in a range of about 5 ppm to about 100 ppm, about 10 ppm to about 1000 ppm, about 50 ppm to about 1500 ppm, or about 500 ppm to about 5,000 ppm. In some embodiments, the hexapeptide-11 is about 1000 ppm. In some embodiments, the hexapeptide-11 is provided at least or about 5, 10, 20, 25, 50, 75, 100, 150, 200, 250, or more than 250 microgram per milliliter (μg/ml). In some embodiments, the hexapeptide-11 is provided in a range of about 25 to about 250, about 50 to about 200, or about 75 to about 150 microgram per milliliter.

In some embodiments, the tripeptide-1 is provided at least or about 0.00001 wt. %, 0.0003 wt. %, 0.0005 wt. %, 0.001 wt. %, 0.001 wt. %, 0.005 wt. %, 0.0055 wt. %, 0.05 wt. %, 0.10 wt. %, 0.25 wt. %, 0.50 wt. %, 0.75 wt. %, 1.0 wt. %, 1.5 wt. %, 2.0 wt. %, 2.5 wt. %, 3.0 wt. %, 3.5 wt. %, 4.0 wt. %, 4.5 wt. %, 5.0 wt. %, 5.5 wt. %, 6.0 wt. %, 6.5 wt. %, 7.0 wt. %, 8 wt. %, 9 wt. %, 10 wt. %, or more than 10 wt. %. In some embodiments, the tripeptide-1 is provided in a range of about 0.25 wt. % to about 10 wt. %, about 0.5 wt. % to about 8 wt. %, about 0.75 wt. % to about 6 wt. %, or about 1 wt. % to about 4 wt. %. In some embodiments, the tripeptide-1 is provided at least or about 0.25, 0.5, 0.75, 1, 1.5, 2, 2.5, 3, 3.5, 4, 5, 6, 7, 8, 9, 10, 15, 20, 25, or more than 25 ppm. In some embodiments, the tripeptide-1 is provided in a range of about 0.25 to about 10, about 0.5 to about 8, about 1 to about 6, or about 2 to about 4 ppm. In some embodiments, the tripeptide-1 is provided in a range of about 1 to about 10 ppm. In some embodiments, the tripeptide-1 is provided at least or about 0.25, 0.5, 0.75, 1, 1.5, 2, 2.5, 3, 3.5, 4, 5, 6, 7, 8, 9, 10, 15, 20, 25, or more than 25 microgram per milliliter (ug/mL). In some embodiments, the tripeptide-1 is provided in a range of about 0.25 to about 10, about 0.5 to about 8, about 1 to about 6, or about 2 to about 4 microgram per milliliter.

In some embodiments, the hexapeptide-12 is provided at least or about 0.00001 wt. %, 0.000 wt. 3%, 0.0005 wt. %, 0.001 wt. %, 0.001 wt. %, 0.005 wt. %, 0.0055 wt. %, 0.05 wt. %, 0.10 wt. %, 0.25 wt. %, 0.50 wt. %, 0.75 wt. %, 1.0 wt. %, 1.5 wt. %, 2.0 wt. %, 2.5 wt. %, 3.0 wt. %, 3.5 wt. %, 4.0 wt. %, 4.5 wt. %, 5.0 wt. %, 5.5 wt. %, 6.0 wt. %, 6.5 wt. %, 7.0%, 8%, 9%, 10%, or more than 10 wt. %. In some embodiments, the hexapeptide-12 is provided in a range of about 0.25 wt. % to about 10 wt. %, about 0.5 wt. % to about 8 wt. %, about 0.75 wt. % to about 6 wt. %, or about 1 wt. % to about 4 wt. %. In some embodiments, the hexapeptide-12 is provided at least or about 0.25, 0.5, 0.75, 1, 1.5, 2, 2.5, 3, 3.5, 4, 5, 6, 7, 8, 9, 10, 15, 20, 25, or more than 25 ppm. In some embodiments, the hexapeptide-12 is provided in a range of about 1 to about 10 ppm. In some embodiments, the hexapeptide-12 is provided in a range of about 0.25 to about 10, about 0.5 to about 8, about 1 to about 6, or about 2 to about 4 ppm. In some embodiments, the hexapeptide-12 is provided at least or about 0.25, 0.5, 0.75, 1, 1.5, 2, 2.5, 3, 3.5, 4, 5, 6, 7, 8, 9, 10, 15, 20, 25, or more than 25 microgram per milliliter (ug/mL). In some embodiments, the hexapeptide-12 is provided in a range of about 0.25 to about 10, about 0.5 to about 8, about 1 to about 6, or about 2 to about 4 microgram per milliliter.

In example embodiments, a weight ratio for the first peptide to the second peptide in a topical formulation is 1 part first peptide to 0.2 to 10 parts second peptide, or 1 to 10 parts second peptide, or 1 to 8 parts second peptide, or 1 to 5.5 parts second peptide. The following nomenclature is employed herein to refer to various amino acids: Alanine (also referred to herein as “Ala” or “A”), Arginine (also referred to herein as “Arg” or “R”), Asparagine (also referred to herein as “Asn” or “N”), Aspartic acid (also referred to herein as “Asp” or “D”), Cysteine (also referred to herein as “Cys” or “C”), Glutamic acid (also referred to herein as “Glu” or “E”), Glutamine (also referred to herein as “Gln” or “Q”), Glycine (also referred to herein as “Gly” or “G”), Histidine (also referred to herein as “His” or “H”), Isoleucine (also referred to herein as “Ile” or “I”), Leucine (also referred to herein as “Leu” or “L”), Lysine (also referred to herein as “Lys” or “K”), Methionine (also referred to herein as “Met” or “M”), Phenylalanine (also referred to herein as “Phe” or “F”), Proline (also referred to herein as “Pro” or “P”), Serine (also referred to herein as “Ser” or “S”), Threonine (also referred to herein as “Thr” or “T”), Tryptophan (also referred to herein as “Trp” or “W”), Tyrosine (also referred to herein as “Tyr” or “Y”), Valine (also referred to herein as “Val” or “V”).

In some embodiments, the first peptide is a dipeptide. Suitable dipeptides include but are not limited to those having the following sequence of amino acids: KK, KP, CK, KC, KT, DF, NF, VW, YR, or TT. In some embodiments, the dipeptide has the following amino acid sequence: KV. In other embodiments, the first peptide is a tripeptide. Suitable tripeptides include but are not limited to those having the following sequence of amino acids: HGG, RKR, GHK, GKH, GGH, GHG, KFK, or KPK. In some embodiments, the tripeptide has the following amino acid sequence: KVK. In some embodiments, the first peptide is a tetrapeptide.

The compositions may include two or more peptides, e.g., two dipeptides and one pentapeptide; one tripeptide and one hexapeptide; one dipeptide, one tripeptide, and one heptapeptide, or the like, provided that the composition contains at least one dipeptide, tripeptide, or tetrapeptide and at least one pentapeptide, hexapeptide, or heptapeptide. In some embodiments, the compositions comprise one or more tripeptides, one or more tetrapeptides, and one or more hexapeptides. In some embodiments, a tripeptide of the one or more tripeptides is tripeptide-1. In some embodiments, a tetrapeptide of the one or more tetrapeptides is tetrapeptide-2. In some embodiments, a hexapeptide of the one or more hexapeptides is hexapeptide-12. In some embodiments, a hexapeptide of the one or more hexapeptides is hexapeptide-11. In some embodiments, the compositions comprise tripeptide-1, tetrapeptide-2, hexapeptide-12, and hexapeptide-11. In some embodiments, the compositions comprise tripeptide-1, tetrapeptide-2, and hexapeptide-12.

The peptide can be functionalized. For example, the peptide can be functionalized with a fatty acid, e.g., myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, docosahexaenoic acid, caprylic acid, capric acid, lauric acid, palmitic acid, stearic acid, arachidic acid, behenic acid, lignoceric acid, cerotic acid, or the like. Examples include palmitoyl hexapeptide-12 (Pal-VGVAPG), palmitoyl tripeptide-1 (Pal-GHK), myristoyl hexapeptide-12 (Myr-VGVAPG), and myristoyl tripeptide-1 (Myr-GHK). Palmitoyl or myristoyl functionalization can be desirable in some embodiments as it exhibits enhanced penetration when compared to other fatty acids. In some embodiments, the peptide is functionalized with a chemical group. For example, the peptide is functionalized with acetyl. In some instances, the peptide is functionalized with a functional group comprising no more than 14 carbons. In some instances, the peptide is functionalized with a functional group comprising no more than 3, 4, 5, 6, 7, 8, 9, 10, 11, 12, 13, 14, 15, 16, 17, 18, 19, 20, or more than 20 carbons. In some instances, the peptide is non-palmitoylated. Without wishing to be limited to a particular theory, incorporation of the peptide in a liposome, in some embodiments, increases the lipophilicity of a peptide that is functionalized or is not functionalized.

Some embodiments of the methods and compositions provided herein include as a first peptide glycine-histidine-lysine (GHK). GHK is a peptide sequence that is rarely found in the class of proteins in general, but is frequently found in extracellular matrix proteins. The small size of GHK permits it to approach membrane receptors far more easily than larger peptides. Further, its unique, copper-binding structure enhances copper transport into and out of cells and promotes wound healing through several different but related pathways. Due to its strong copper binding structure, GHK can be provided in the form of GHK-Cu (copper-bound GHK form).

GHK acts as an anti-inflammatory (see, e.g., Pickart, L., The human tri-peptide GHK and tissue remodeling, J. Biomater. Sci. Polymer Edn. 2008, Vol. 19, pp. 969-988, 972-973; Pickart et al., The Human Tripeptide GHK-CU in Prevention of Oxidative Stress and Degenerative Conditions of Aging: Implications for Cognitive Health, Oxid. Med. Cell Longev. 2012, Vol. 2012, pp. 1-8, 3) and an antioxidant. GHK acts to promote wound healing by suppressing the “acute phase response” that can produce both inflammation and induce scarring. This biological response prevents the invasion of bacteria, facilitates the arrival of immune cells, stems bleeding, and provides a covering for the wounded area. GHK-Cu also suppresses the acute phase response by inhibiting the production of molecules called cytokines. Cytokines are immune cell signaling molecules that attract immune cells and that trigger the production of other molecules that promote inflammation and fibrosis (leading to the creation of scar tissue). In particular, GHK suppresses the production of cytokines including tumor necrosis factor-alpha (TNFα), interleukin-1 (IL-1), interleukin-6 (IL-6), and transforming growth factor-beta-1 (TGF-β1), a few of the key drivers of inflammation and apoptotic cell death in the wound region. As TGF-β1 is an important component for the continuation of the acute phase response, GHK's suppression of TGF-β1 also acts to shorten the duration of the acute phase response once it has begun. GHK acts as an antioxidant by blocking ferritin's release of oxidizing iron, preventing further inflammation or microbial infection (as invading microbes need iron to survive).

GHK also stimulates blood vessel growth, increases collagen production, and regenerates the extracellular matrix. GHK acts as an attractant for cells vital to the regeneration of damaged tissues such as capillary cells that rebuild blood vessels. It also upregulates the production of a variety of enzymes that remove damaged proteins while also rebuilding the extracellular matrix (ECM), a key external scaffold that is important for intercellular communication and support. In particular, GHK's induces the production of messenger RNAs (mRNAs) necessary for the regeneration of the ECM, namely collagen, proteoglycans, glycosaminoglycans, chondroitin sulfate, and dermatan sulfate. GHK's induction of increased collagen production also plays a key role in enhancing skin regrowth. GHK further stimulates blood flow into damaged tissues through three processes: angiogenesis, anti-coagulation and vascular dilation. First, GHK induces angiogenesis or new blood vessel formation by increasing the production of growth factor proteins necessary for angiogenesis such as basic fibroblast growth factor (BFGF) and vascular endothelial growth factor (VEGF). Second, GHK increases blood flow to the wounded area by expanding the number of red blood cells (via growth in erythropoietin production) and by anti-coagulatory effects such as downregulating the blood clotting molecule thromboxane. Third, GHK facilitates vascular dilation through binding to the vasoconstriction protein angiotensin II, preventing angiotensin from constricting blood vessels and reducing blood flow.

GHK promotes stem cell proliferation (see, e.g., Ito et al., Is the Hair Follicle Necessary for Normal Wound Healing, J. Invest. Dermatol. 2008, Vol. 128, pp. 1059-1061, 1059). Wound healing studies have demonstrated that the addition of GHK-Cu greatly enlarged the production of hair follicles near the wound periphery in experiments with mice. Dermal hair follicles are a significant source of stem cells that are essential for dermal healing. Research into dermal hair follicles have demonstrated that hair-bearing areas tend to heal more quickly and that cells from various portions of the follicle may contribute to both dermal cell and epithelial cell replacement as well.

Thus, by decreasing inflammation, acting as an antioxidant, stimulating growth of new blood vessels, regenerating the extracellular matrix, enhancing collagen production, and by promoting stem cell proliferation, GHK can greatly enhance skin regeneration and promote wound healing.

Some embodiments of the methods and compositions provided herein include as a second peptide valine-glycine-valine-alanine-proline-glycine (VGVAPG). VGVAPG is a hexapeptide that is derived from the elastin protein (see, e.g., Blanchevoye et al., Interaction between the Elastin Peptide VGVAPG and Human Elastin Binding Protein, J. Biol. Chem. 2012, Vol. 288, pp. 1317-1328, 1317-1318) (“VGVAPG” disclosed as SEQ ID NO: 9). Elastin is a protein found in connective tissue (e.g. skin) that is necessary for tissues to return to their original shape and size after undergoing temporary expansion or contraction. Due to the importance of elastin in providing elasticity and resilience, elastin plays a significant role in skin cell resistance to injury and recovery from injury. The ability of skin to return to its original form after undergoing stretching or pulling relies on cross-linked elastin proteins (tropoelastin proteins in humans) that work to form “elastic fibers.” The disruption of the elastic fiber system in healing wounds has been strongly linked to the production of scar tissue (see, e.g., Rnjak-Kovacina et al., Severe Burn Injuries and the Role of Elastin in the Design of Dermal Substitutes, Tissue Eng. Part B. Rev. 2011, pp. 81-91, 85-86). Because of these properties and others, elastin is a key component in the effective wound healing process.

VGVAPG plays a role in facilitating elastin's ability to prevent skin injury and to promote skin regeneration (see, e.g., Floquet et al., Structural Characterization of VGVAPG, an Elastin-Derived Peptide, Biopolymers (Peptide Science) 2004, Vol. 76, 266-280, 267) (“VGVAPG”). First, it has been shown to demonstrate the ability to attract monocytes and fibroblasts (see, e.g., Senior et al., Val-Gly-Val-Ala-Pro-Gly, a Repeating Peptide in Elastin, Is Chemotactic for Fibroblasts and Monocytes, J. Cell Biol. 1984, Vol. 99, pp. 870-874, 870) (“Val-Gly-Val-Ala-Pro-Gly”), monocytes being essential for fighting off infection and fibroblasts being necessary for collagen production (the most abundant protein in skin) and for the regeneration of the extracellular matrix. Second, VGVAPG provides a binding site for elastin-binding protein, a permanent component of mature elastic fibers. Third, VGVAPG provides a binding site for elastin and extracellular matrix degradation enzymes such as matrix metalloproteinases (MMPs), which facilitate the replacement and regeneration of elastic fibers and extracellular matrix proteins.

The tripeptide and hexapeptide work synergistically to promote skin regeneration and wound healing through the attraction of healing cells, increased production of elastin and collagen, enhanced fibroblast proliferation, antioxidant behavior (preventing the release of oxidizing iron), and inducing the regeneration of the extracellular matrix. As a result, the combination of the two peptides exhibits synergistic, superior performance well beyond that expected for either of the two peptides alone.

Tripeptides promote skin regeneration through increased collagen and elastin synthesis, blocking ferritin release of oxidized iron, attracting healing cells such as capillary cells and macrophages, and through re-establishing new blood flow to the injury site. The tripeptide functions as an anti-oxidant, stimulates collagen, elastin, and hyaluronic acid. It is formulated to penetrate stratum corneum. In the extracellular matrix (ECM), it is an anti-oxidant, attracts capillaries and macrophages, which facilitates wound healing. In the cell, it decreases inflammatory cytokines, increases collagen, elastin, dermal stem cell proliferation, and hyaluronic acid.

Hexapeptides promote skin regeneration and wound healing through the induction of elastin and collagen production, fibroblast proliferation, regeneration of the extracellular matrix, and fibroblast keratinocyte mobility. The hexapeptide is formulated to penetrate the stratum corneum, and mimics the elastin binding sequence, to stimulate elastin. It binds specifically to EBP receptors on fibroblasts and keratinocytes. The binding initiates intracellular signal transduction.

The peptides can advantageously be provided in a base for suitable for combining with other components of a topical formulation. The base can include one or more components such as a thickener/binding agent (e.g., pentaerythrityl tetraisostearate), an emollient/dispersing agent (e.g., caprylic/capric triglyceride), a solvent (e.g., propylene carbonate), and/or a rheology modifier/antisettling agent (e.g., disteardimonium hectorite).

Liposomes

Described herein are liposomal compositions for improved distribution, efficacy, bioavailability, and/or activity. Liposomal compositions may improve distribution, efficacy, bioavailability, and/or activity of the active ingredient by improving delivery and tissue (e.g. skin) penetration. In some instances, improved delivery and skin penetration result from the active ingredient being incorporated (e.g. encapsulated) in a liposome. In some instances, the active ingredient is a peptide that is encapsulated in a liposome.

Liposomal compositions as described herein may comprise a peptide encapsulated in a liposome. In some embodiments, the peptide is tripeptide-1. In some embodiments, the peptide is hexapeptide-12. In some embodiments, the peptide is hexapeptide-11 In some embodiments, the peptide is tetrapeptide-2. In some embodiments, the peptide is functionalized with a palmitoyl group. In some embodiments, the peptide is functionalized with an acetyl group. For example, the peptide is acetyl hexapeptide-38.

Liposomal compositions as described herein may comprise various ingredients encapsulated in a liposome. In some embodiments, the ingredient is lactoferrin.

Lecithin and other phospholipids may be used to prepare liposomes containing the peptide compositions as described herein. In some embodiments, liposomes are used to prepare one or more peptides. In some embodiments, the peptide is functionalized with an acetyl group. Formation of lipid vesicles occurs when phospholipids such as lecithin are placed in water and consequently form one bilayer or a series of bilayers, each separated by water molecules, once enough energy is supplied. Liposomes can be created by sonicating phospholipids in water. Low shear rates create multilamellar liposomes. Continued high-shear sonication tends to form smaller unilamellar liposomes. Hydrophobic chemicals can be dissolved into the phospholipid bilayer membrane. The lipid bilayers of the liposomes deliver the peptide compositions as described herein.

The phospholipids used to prepare the liposomal compositions described herein may comprise a transition phase temperature of about 10° C. to about 25° C. In some instances, the phospholipids comprise a transition phase temperature of about 10° C., 12° C., 14° C., 16° C., 18° C., 20° C., 22° C., 24° C., 26° C., 28° C., 30° C., 32° C., 34° C., 36° C., 38° C., 40° C., or more than 40° C. In some instances, the phospholipids comprise a transition phase temperature in a range of about 10° C. to about 40° C., about 12° C. to about 36° C., about 14° C. to about 32° C., about 16° C. to about 20° C., or about 21° C. to about 25° C.

The topical composition may contain micelles, or an aggregate of surfactant molecules dispersed in an aqueous solution. Micelles may be prepared by dispersing an oil solvent in an aqueous solution comprising a surfactant, where the surfactant concentration exceeds the critical micelle concentration. The resulting composition contains micelles, i.e., spherical oil droplets.

The liposomal composition may contain micelles, or an aggregate of surfactant molecules dispersed in an aqueous solution. Micelles may be prepared by dispersing an oil solvent in an aqueous solution comprising a surfactant, where the surfactant concentration exceeds the critical micelle concentration. The resulting formulation contains micelles, i.e., spherical oil droplets surrounded by a membrane of polar surfactant molecules, dispersed in the aqueous solvent.

Described herein, in some embodiments, are methods for preparing a composition comprising a peptide encapsulated in a liposome, comprising: combining the peptide and a solvent to form a mixture; and contacting the mixture with an aqueous solution comprising liposomes. In some instances, the contacting occurs at a temperature between about 10° C. and about 25° C. In some instances, the contacting occurs at a temperature of about 10° C., 12° C., 14° C., 16° C., 18° C., 20° C., 22° C., 24° C., 26° C., 28° C., 30° C., 32° C., 34° C., 36° C., 38° C., 40° C., or more than 40° C. In some instances, the contacting occurs at a temperature in a range of about 10° C. to about 40° C., about 12° C. to about 36° C., about 14° C. to about 32° C., about 16° C. to about 20° C., or about 21° C. to about 25° C.

Methods for preparing a composition comprising a peptide encapsulated in a liposome may comprise use of a solvent. In some instances, the solvent is water. In some instances, the solvent is an organic solvent. Exemplary organic solvents include, but are not limited to, petroleum ether, cyclohexane, toluene, carbon tetrachloride, dichloromethane, chloroform, diethyl ether, diisopropyl ether, ethyl acetate, butanol, n-propanol, ethanol, methanol, polyethylene glycol, propylene glycol, and pyridine. In some instances, the solvent is a glycol. In some instances, the solvent is butylene glycol. In some instances, the solvent is caprylyl glycol. In some instances, the solvent is propanediol (propylene glycol).

The solvent may be used at various percentages. In some instances, the solvent is provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10%. The solvent may be propanediol, butylene glycol, or caprylyl glycol.

Methods as described herein, in some embodiments, comprises combining the peptide and a solvent to form a mixture; and contacting the mixture with an aqueous solution comprising liposomes, wherein the aqueous solution comprises a percentage of water and a percentage of liposomes. In some instances, the aqueous solution comprises at least or about 20%, 30%, 40%, 50%, 60%, 70%, 80%, 90%, or more than 90% water. In some instances, the aqueous solution comprises water in a range of about 10% to about 95%, about 20% to about 90%, about 30% to about 85%, about 40% to about 80%, or about 50% to about 60%. In some instances, the aqueous solution comprises at least or about 20%, 30%, 40%, 50%, 60%, or more than 60% liposomes. In some instances, the aqueous solution comprises liposomes in a range of about 10% to about 80%, about 20% to about 70%, or about 30% to about 60%. A ratio of liposomes to water may be in a range of about 1:9 to about 3:7. In some instances, the ratio of liposomes to water may be at least or about 1:10, 1:9, 1:8, 1:7, 1:6, 1:5, 1:4, 1:3, or 1:2.

Methods for generation of liposomal compositions as described herein may result in an entrapment efficacy of no more than 100%. In some instances, the entrapment efficacy is no more than 50%, 60%, 70%, 80%, 90%, 95%, 99%, or 99.5%.

Described herein are liposomal compositions, wherein the peptide comprises a percentage of the composition. In some embodiments, the peptide is provided at least or about 0.0001%, 0.0005%, 0.00055%, 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% of the composition. In some embodiments, the peptide is provided at least or about 10%, 11%, 12%, 13%, 14%, 15%, 16%, 17%, 18%, 19%, 20%, 22%, 24%, 26%, 28%, 30% or more than 30% of the composition. In some embodiments, the peptide is provided in a range of about 0.001% to about 6%, about 0.002% to about 4%, about 0.01% to about 5%, or about 0.02% to about 2% by weight. In some embodiments, the peptide is provided at about 0.03% of the composition.

Described herein are liposomal compositions, wherein the liposomes comprise a percentage of the composition. In some embodiments, the liposomes are provided at least or about 10%, 11%, 12%, 13%, 14%, 15%, 16%, 17%, 18%, 19%, 20%, 22%, 24%, 26%, 28%, 30% or more than 30% of the composition. In some embodiments, the liposomes are provided in a range of about 5% to about 90%, about 10% to about 80%, about 20% to about 70%, about 30% to about 60%, about 10% to about 30%, or about 20% to about 40%.

Liposomal compositions as described herein, in some embodiments, comprise an average particle size of at most 220 nanometers (nm). In some instances, the average particle size is at most 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, 205 nm, 210 nm, 215 nm, 220 nm, 230 nm, 240 nm, 250 nm, 260 nm, 270 nm, 280 nm, 290 nm, 300 nm, 320 nm, 340 nm, 360 nm, 380 nm, or 400 nm. In some instances, the average particle size is about 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, 205 nm, 210 nm, 215 nm, 220 nm, 230 nm, 240 nm, 250 nm, 260 nm, 270 nm, 280 nm, 290 nm, 300 nm, 320 nm, 340 nm, 360 nm, 380 nm, or 400 nm. In some instances, the average particle size is in a range of about 50 nm to about 500 nm, about 100 nm to about 400 nm, about 150 nm to about 220 nm, about 180 nm to about 220 nm, or about 190 nm to about 210 nm.

In some instances, the liposomal compositions comprise an active agent that has a molecular weight of no more than about 600 Daltons (Da). In some instances, the active agent has a molecular weight of at least or about 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, or more than 1000 Daltons (Da). In some instances, the active agent has a molecular weight of at least or about 1000, 1100, 1200, 1300, 1400, 1500, 1600, 1700, 1800, 1900, 2000, 2100, 2200, 2300, 2400, 2500, 2600, 2700, 2800, 2900, 3000, 4000, 5000, 6000, or more than 6000 Daltons (Da). In some instances, the active agent has a molecular weight in a range of about 50 to about 1000, about 100 to about 900, about 200 to about 800, about 300 to about 700, or about 400 to about 600 Daltons (Da). In some instances, the active agent is a peptide. In some instances, the active agent is a peptide encapsulated in a liposome.

A polydispersity index (PdI) of a liposomal composition as described herein, in some embodiments, is in a range of 0 to about 0.2. In some instances, the polydispersity index is about 0.01, 0.025, 0.05, 0.1, 0.25, 0.3, 0.35, 0.4, 0.45, 0.5, 0.55, 0.6, 0.65, 0.7, 0.75, or 0.8. In some instances, the polydispersity index is in a range of about 0.01 to about 0.8, about 0.025 to about 0.75, about 0.05 to about 0.6, or about 0.1 to about 0.3.

In some instances, an intercept of a liposomal composition as described herein is in a range of about 0.85 to about 0.95. In some instances, the intercept is the amplitude. In some instances, the intercept is at least or about 0.65, 0.70, 0.75, 0.80, 0.85, 0.90, or 0.95.

In some embodiments, the liposomes comprise propanediol, lecithin, or a combination thereof. In some embodiments, the propanediol is provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt. %). In some embodiments, the propanediol is provided in a range of about 0.001% to about 6%, about 0.002% to about 4%, about 0.01% to about 3%, or about 0.02% to about 2% by weight. In some embodiments, the lecithin is provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt. %). In some embodiments, the lecithin is provided in a range of about 0.001% to about 6%, about 0.002% to about 4%, about 0.01% to about 3%, or about 0.02% to about 2% by weight. In some embodiments, the liposomes comprise propanediol and lecithin. In some embodiments, the propanediol and lecithin are provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, 4.5%, 5.0%, 5.5%, 6.0%, 6.5%, 7.0%, 8%, 9%, 10%, or more than 10% by weight (wt. %). In some embodiments, the propanediol and lecithin are provided in a range of about 0.001% to about 6%, about 0.002% to about 4%, about 0.01% to about 3%, or about 0.02% to about 2% by weight. In some embodiments, the propanediol and lecithin are provided at about 0.90% by weight

Described herein are liposomal compositions comprising improved distribution, efficacy, bioavailability, and/or activity. The liposomal compositions may comprise improved distribution, efficacy, bioavailability, and/or activity as compared to compositions not comprising liposomes. In some instances, the distribution is improved by at least or about 0.5×, 1.0×, 1.5×, 2.0×, 2.5×, 3.0×, 4.0×, 4.5×, 5×, or more than 5× as compared to compositions not comprising liposomes. In some instances, the efficacy is improved by at least or about 0.5×, 1.0×, 1.5×, 2.0×, 2.5×, 3.0×, 4.0×, 4.5×, 5×, or more than 5× as compared to compositions not comprising liposomes. In some instances, the bioavailability is improved by at least or about 0.5×, 1.0×, 1.5×, 2.0×, 2.5×, 3.0×, 4.0×, 4.5×, 5×, or more than 5× as compared to compositions not comprising liposomes. In some instances, the activity is improved by at least or about 0.5×, 1.0×, 1.5×, 2.0×, 2.5×, 3.0×, 4.0×, 4.5×, 5×, or more than 5× as compared to compositions not comprising liposomes. The distribution, efficacy, bioavailability, and/or activity may be improved by at least or about 10%, 15%, 20%, 25%, 30%, 35%, 40%, 45%, 50%, 55%, 60%, 65%, 70%, 75%, 80%, 85%, 90%, or more than 90% as compared to compositions not comprising liposomes.

Liposomal compositions and methods as described herein, in some embodiments, are topical compositions. In some instances, the liposomal compositions are oil free. In some instances, the liposomal compositions are preservative free. In some embodiments, the liposomal formulation is an aqueous formulation. In some embodiments, the liposomal formulation is an anhydrous formulation. In some instances, the liposomal composition comprises a pH in a range of about 5 to about 8. In some instances, the liposomal composition comprises a pH of at least or about 2, 3, 4, 5, 6, 7, 8, 9, or 10.

Methods and compositions as described herein may result in improved follicular penetration. In some instances, the follicular penetration is improved by at least or about 0.5×, 1.0×, 1.5×, 2.0×, 2.5×, 3.0×, 4.0×, 4.5×, 5×, or more than 5×. The follicular penetration may be improved by at least or about 10%, 15%, 20%, 25%, 30%, 35%, 40%, 45%, 50%, 55%, 60%, 65%, 70%, 75%, 80%, 85%, 90%, or more than 90%. In some instances, compositions result in follicular penetration of a depth of at least or about 0.5, 0.75, 1, 1.25, 1.5, 2, 2.5, 3, 3.5, 4, 4.5, 5, 5.5, 6, 7, 8, 9, 10, or more than 10 millimeters.

Peucedanum graveolens

Peucedanum graveolens is an herb commonly known as dill. Compositions as described herein, in some embodiments, comprise Peucedanum graveolens extract. In some embodiments, Peucedanum graveolens extract is provided at least or about 0.005 wt. %, 0.01 wt. %, 0.02 wt. %, 0.05 wt. %, 0.10 wt. %, 0.20 wt. %, 0.25 wt. %, 0.50 wt. %, 0.75 wt. %, 1.0 wt. %, 1.5 wt. %, 2.0 wt. %, 2.5 wt. %, 3.0 wt. %, 3.5 wt. %, 4.0 wt. %, or more than 4 wt. %. In some embodiments, the Peucedanum graveolens extract is provided in a range of about 0.25 wt. % to about 10 wt. %, about 0.1 wt. % to about 2.5 wt. %, about 0.5 wt. % to about 8 wt. %, about 0.75 wt. % to about 6 wt. %, or about 1 wt. % to about 4 wt. %. In some embodiments, the Peucedanum graveolens extract is provided in a range of about 0.001 wt. % to about 6 wt. %, about 0.002 wt. % to about 4 wt. %, about 0.01 wt. % to about 3 wt. %, about 0.01 wt. % to about 2.5 wt. %, or about 0.02 wt. % to about 2 wt. %. In some embodiments, the Peucedanum graveolens extract comprises about 0.5 wt. %.

Lactoferrin

Lactoferrin (Lf) is an 80 kDa iron binding glycoprotein of the transferrin family, found in exocrine secretions (tears, saliva, milk, nasal and bronchial secretions, gastrointestinal fluids and others). Lactoferrin effects range from antimicrobial to anti-inflammatory and immune modulator activities with high iron binding affinity. Lactoferrin can downregulate TNFα and other cytokine production (IL-1) by local skin cells and may be involved bruising resolution and in the prevention of post inflammatory pigmentation. Lactoferrin can also have a positive effect on wound healing.

Compositions as described herein, in some embodiments, comprise a glycoprotein. In some embodiments, glycoprotein is provided at least or about 0.001 wt. %, 0.005 wt. %, 0.01 wt. %, 0.02 wt. %, 0.05 wt. %, 0.10 wt. %, 0.20 wt. %, 0.25 wt. %, 0.50 wt. %, 0.75 wt. %, 1.0 wt. %, 1.5 wt. %, 2.0 wt. %, 2.5 wt. %, 3.0 wt. %, 3.5 wt. %, 4.0 wt. %, or more than 4 wt. %. In some embodiments, the glycoprotein is provided in a range of about 0.005 wt. % to about 0.1 wt. %, about 0.25 wt. % to about 10 wt. %, about 0.5 wt. % to about 8 wt. %, about 0.75 wt. % to about 6 wt. %, or about 1 wt. % to about 4 wt. %. In some embodiments, the glycoprotein is provided in a range of about 0.001 wt. % to about 6 wt. %, about 0.002 wt. % to about 4 wt. %, about 0.01 wt. % to about 2.5 wt. %, or about 0.02 wt. % to about 2 wt. %. In some embodiments, the glycoprotein is provided in a range of about 0.01 wt. % to about 0.25 wt. %. In some embodiments, the glycoprotein is provided at about 0.025 wt. %. In some embodiments, the glycoprotein is provided at about 0.05 wt. %. In some embodiments, the glycoprotein is provided at about 0.10 wt. %. In some embodiments, the glycoprotein is provided at least or about 5, 10, 20, 25, 50, 75, 100, 150, 200, 250, 300, 350, 400, 450, 500, 550, 600, 650, 700, 750, 800, 850, 900, 950, 1000 or more than 1000 microgram per milliliter (μg/ml). In some embodiments, the glycoprotein is provided in a range of about 5 to about 1000, about 10 to about 900, about 20 to about 800, about 25 to about 700, about 50 to about 600, or about 75 to about 500 microgram per milliliter (μg/ml). In some embodiments, the glycoprotein is provided at about 100 μg/ml. In some embodiments, the glycoprotein is provided at least of about 100 part per million (ppm), 200 ppm, 300 ppm, 400 ppm, 500 ppm, 600 ppm, 700 ppm, 800 ppm, 900 ppm, 1000 ppm, 1100 ppm, 1200 pm, 1300 ppm, 1400 ppm, 1500 ppm, 1600 ppm, 1700 ppm, 1800 ppm, 1900 ppm, 2000 ppm, or more than 2000 ppm. In some embodiments, the glycoprotein is provided in a range 100 ppm to about 1900 ppm, about 200 ppm to about 1800 ppm, about 200 ppm to about 1700 ppm, about 400 ppm, to about 1600 ppm, about 500 ppm to about 1500 ppm, about 600 ppm to about 1400 ppm, about 700 ppm to about 1300 ppm, about 800 ppm to about 1200 ppm, or about 900 ppm to about 1100 ppm. In some embodiments, the glycoprotein is provided in a range of about 10 ppm to 1000 ppm, in a range of about 50 ppm to about 1000 ppm, about 100 ppm to about 1000 ppm, or about 500 ppm to about 1000 ppm.

Compositions as described herein, in some embodiments, comprise a transferrin. In some embodiments, transferrin is provided at least or about 0.001 wt. %, 0.005 wt. %, 0.01 wt. %, 0.02 wt. %, 0.05 wt. %, 0.10 wt. %, 0.20 wt. %, 0.25 wt. %, 0.50 wt. %, 0.75 wt. %, 1.0 wt. %, 1.5 wt. %, 2.0 wt. %, 2.5 wt. %, 3.0 wt. %, 3.5 wt. %, 4.0 wt. %, or more than 4 wt. %. In some embodiments, the transferrin is provided in a range of about 0.005 wt. % to about 0.1 wt. %, about 0.25 wt. % to about 10 wt. %, about 0.5 wt. % to about 8 wt. %, about 0.75 wt. % to about 6 wt. %, or about 1 wt. % to about 4 wt. %. In some embodiments, the transferrin is provided in a range of about 0.001 wt. % to about 6 wt. %, about 0.002 wt. % to about 4 wt. %, about 0.01 wt. % to about 2.5 wt. %, or about 0.02 wt. % to about 2 wt. %. In some embodiments, the transferrin is provided in a range of about 0.01 wt. % to about 0.25 wt. %. In some embodiments, the transferrin is provided at about 0.025 wt. %. In some embodiments, the transferrin is provided at about 0.05 wt. %. In some embodiments, the transferrin is provided at about 0.10 wt. %. In some embodiments, the transferrin is provided at least or about 5, 10, 20, 25, 50, 75, 100, 150, 200, 250, 300, 350, 400, 450, 500, 550, 600, 650, 700, 750, 800, 850, 900, 950, 1000 or more than 1000 microgram per milliliter (μg/ml). In some embodiments, the transferrin is provided in a range of about 5 to about 1000, about 10 to about 900, about 20 to about 800, about 25 to about 700, about 50 to about 600, or about 75 to about 500 microgram per milliliter (μg/ml). In some embodiments, the transferrin is provided at about 100 μg/ml. In some embodiments, the transferrin is provided at least of about 100 part per million (ppm), 200 ppm, 300 ppm, 400 ppm, 500 ppm, 600 ppm, 700 ppm, 800 ppm, 900 ppm, 1000 ppm, 1100 ppm, 1200 pm, 1300 ppm, 1400 ppm, 1500 ppm, 1600 ppm, 1700 ppm, 1800 ppm, 1900 ppm, 2000 ppm, or more than 2000 ppm. In some embodiments, the transferrin is provided in a range 100 ppm to about 1900 ppm, about 200 ppm to about 1800 ppm, about 200 ppm to about 1700 ppm, about 400 ppm, to about 1600 ppm, about 500 ppm to about 1500 ppm, about 600 ppm to about 1400 ppm, about 700 ppm to about 1300 ppm, about 800 ppm to about 1200 ppm, or about 900 ppm to about 1100 ppm. In some embodiments, the transferrin is provided in a range of about 10 ppm to 1000 ppm, in a range of about 50 ppm to about 1000 ppm, about 100 ppm to about 1000 ppm, or about 500 ppm to about 1000 ppm.

In some embodiments, the transferrin is a lactoferrin. In some embodiments, the composition comprises a trypsinized fragment of lactoferrin. In some embodiments, the compositions comprise lactoferrin. In some embodiments, the compositions comprise a variant or fragment of lactoferrin.

In some instances, the lactoferrin has antimicrobial activity. In some instances, the lactoferrin has antimicrobial activity against bacteria, fungi, yeasts, viruses, parasites, or combinations thereof. Lactoferrin, in some instances, comprises antibiofilm activity. In some instances, lactoferrin interacts with the bacterial surface and destabilizes the microbial membrane. In some instances, lactoferrin chelates iron to disrupt the microbial membrane. In some embodiments, the lactoferrin has synergistic effects with xylitol to exert antimicrobial activity, antibiofilm activity, or both.

In some embodiments, lactoferrin is provided at least or about 0.001 wt. %, 0.005 wt. %, 0.01 wt. %, 0.02 wt. %, 0.05 wt. %, 0.10 wt. %, 0.20 wt. %, 0.25 wt. %, 0.50 wt. %, 0.75 wt. %, 1.0 wt. %, 1.5 wt. %, 2.0 wt. %, 2.5 wt. %, 3.0 wt. %, 3.5 wt. %, 4.0 wt. %, or more than 4 wt. %. In some embodiments, the lactoferrin is provided in a range of about 0.005 wt. % to about 0.1 wt. %, about 0.25 wt. % to about 10 wt. %, about 0.5 wt. % to about 8 wt. %, about 0.75 wt. % to about 6 wt. %, or about 1 wt. % to about 4 wt. %. In some embodiments, the lactoferrin is provided in a range of about 0.001 wt. % to about 6 wt. %, about 0.002 wt. % to about 4 wt. %, about 0.01 wt. % to about 2.5 wt. %, or about 0.02 wt. % to about 2 wt. %. In some embodiments, the lactoferrin is provided in a range of about 0.01 wt. % to about 0.25 wt. %. In some embodiments, the lactoferrin is provided at about 0.025 wt. %. In some embodiments, the lactoferrin is provided at about 0.05 wt. %. In some embodiments, the lactoferrin is provided at about 0.10 wt. %. In some embodiments, the lactoferrin is provided at least or about 5, 10, 20, 25, 50, 75, 100, 150, 200, 250, 300, 350, 400, 450, 500, 550, 600, 650, 700, 750, 800, 850, 900, 950, 1000 or more than 1000 microgram per milliliter (μg/ml). In some embodiments, the lactoferrin is provided in a range of about 5 to about 1000, about 10 to about 900, about 20 to about 800, about 25 to about 700, about 50 to about 600, or about 75 to about 500 microgram per milliliter (μg/ml). In some embodiments, the lactoferrin is provided at about 100 μg/ml. In some embodiments, the lactoferrin is provided at least of about 100 part per million (ppm), 200 ppm, 300 ppm, 400 ppm, 500 ppm, 600 ppm, 700 ppm, 800 ppm, 900 ppm, 1000 ppm, 1100 ppm, 1200 pm, 1300 ppm, 1400 ppm, 1500 ppm, 1600 ppm, 1700 ppm, 1800 ppm, 1900 ppm, 2000 ppm, or more than 2000 ppm. In some embodiments, the lactoferrin is provided in a range 100 ppm to about 1900 ppm, about 200 ppm to about 1800 ppm, about 200 ppm to about 1700 ppm, about 400 ppm, to about 1600 ppm, about 500 ppm to about 1500 ppm, about 600 ppm to about 1400 ppm, about 700 ppm to about 1300 ppm, about 800 ppm to about 1200 ppm, or about 900 ppm to about 1100 ppm. In some embodiments, the lactoferrin is provided in a range of about 10 ppm to 1000 ppm, in a range of about 50 ppm to about 1000 ppm, about 100 ppm to about 1000 ppm, or about 500 ppm to about 1000 ppm.

Oleuropein

In some embodiments, polyphenols such as oleuropein may be added to the compositions. Oleuropein is a polyphenol isolated from olive leaves (see e.g. Omar S H. Oleuropein in olive and its pharmacological effects. Sci Pharm 2010; 78(2): 133-54; Al-Rimawi F, Yateem H, Afaneh I. Formulation and evaluation of a moisturizing day cream containing olive leaves extract. International Journal of Development Research 2014; 4(10): 1996-2000; Kontogianni V G, Charisiadis P, Margianni E, Lamari F N, Gerothanassis I P, Tzakos A G. Olive leaf extracts are a natural source of advanced glycation end product inhibitors. Journal of medicinal food 2013; 16(9): 817-22). Oleuropein demonstrates major anti-inflammatory effects by inhibiting lipoxygenase activity and the production of leukotriene. More particularly researchers have demonstrated that oleuropein enhances proteasome activities in vitro more effectively than other known chemical activators, possibly through conformational changes of the proteasome. In this regard, it decreases reactive oxygen species (ROS), reduces the amount of oxidized proteins through increased proteasome-mediated degradation through increased proteasome-mediated degradation and autophagic pathways, and retains proteasome function during replicative senescence. Inhibition of AGE formation via blocking sugar attachment to proteins, scavenging the reactive intermediates, or breakdown of established AGE-induced cross-links constitutes an attractive therapeutic/preventive target. Oleuropein has been demonstrated to inhibit AGE formation and breakdown AGE products through its proteasome enhancing function. When oleuropein is employed in a topical formulation, it is preferably present at from about 0.005% by weight or less to about 10.0% by weight or more, typically at from about 0.01% by weight to about 5.0% by weight, e.g., at from about 0.05% by weight to about 0.1% by weight. Oleuropein is useful in compositions for promoting healing. Oleuropein is typically not employed in antiaging compositions, in that its effects tend to be incompatible with volumizing, but it can advantageously be employed in formulations for preconditioning the skin in advance of procedures as described herein (e.g., laser resurfacing, chemical peel, etc.).

Compositions as described herein, in some embodiments, comprise oleuropein. In some instances, oleuropein is present at about 50 ppm or less to 1000, 5000, 10000, 50000, 100000, 500000 ppm or more, e.g., 100 ppm of the oleuropein. In some instances, oleuropein is present at about 1, 2, 3, 4, 5, 6, 7, 8, 9, 10, 15, 20, 25, 50, 75, 100, 200, 300, 400, 500, 600, 700, 800, 900, 1000, or more than 1000 ppm. In some instances, oleuropein is present in a range of about 1 to about 100, about 1 to about 50, about 1 to about 40, about 1 to about 30, about 1 to about 20, about 1 to about 10, about 5 to about 90, about 10 to about 80, about 20 to about 60, or about 30 to about 50 ppm. In some instances, oleuropein is present at about 1, 2, 3, 4, 5, 6, 7, 8, 9, 10, 15, 20, 25, 50, 75, 100, 200, 300, 400, 500, 600, 700, 800, 900, 1000, or more than 1000 microgram per milliliter (ug/mL). In some instances, oleuropein is present in a range of about 1 to about 100, about 1 to about 50, about 1 to about 40, about 1 to about 30, about 1 to about 20, about 1 to about 10, about 5 to about 90, about 10 to about 80, about 20 to about 60, or about 30 to about 50 microgram per milliliter. In some instances, oleuropein is present from about 0.01 wt. % to about 10 wt. %, about 0.01 wt. % to about 0.02 wt. %, about 0.01 wt. % to about 0.03 wt. %, about 0.01 wt. % to about 0.04 wt. %, about 0.01 wt. % to about 0.05 wt. %, about 0.01 wt. % to about 0.1 wt. %, about 0.01 wt. % to about 0.25 wt. %, about 1 wt. % to about 5 wt. %, or about 1 wt. % to about 10 wt. %. In some instances, oleuropein is present about 0.05 wt. %.

Compositions as described herein, in some embodiments, comprise Olea europaea leaf extract. In some instances, oleuropein is present at about 50 ppm or less to 1000, 5000, 10000, 50000, 100000, 500000 ppm or more, e.g., 100 ppm of the Olea europaea leaf extract. In some instances, Olea europaea leaf extract is present at about 1, 2, 3, 4, 5, 6, 7, 8, 9, 10, 15, 20, 25, 50, 75, 100, 200, 300, 400, 500, 600, 700, 800, 900, 1000, or more than 1000 ppm. In some instances, Olea europaea leaf extract is present in a range of about 1 to about 100, about 1 to about 50, about 1 to about 40, about 1 to about 30, about 1 to about 20, about 1 to about 10, about 5 to about 90, about 10 to about 80, about 20 to about 60, or about 30 to about 50 ppm. In some instances, Olea europaea leaf extract is present at about 1, 2, 3, 4, 5, 6, 7, 8, 9, 10, 15, 20, 25, 50, 75, 100, 200, 300, 400, 500, 600, 700, 800, 900, 1000, or more than 1000 microgram per milliliter (ug/mL). In some instances, Olea europaea leaf extract is present in a range of about 1 to about 100, about 1 to about 50, about 1 to about 40, about 1 to about 30, about 1 to about 20, about 1 to about 10, about 5 to about 90, about 10 to about 80, about 20 to about 60, or about 30 to about 50 microgram per milliliter. In some instances, Olea europaea leaf extract is present from about 0.01 wt. % to about 10 wt. %, about 0.01 wt. % to about 0.02 wt. %, about 0.01 wt. % to about 0.03 wt. %, about 0.01 wt. % to about 0.04 wt. %, about 0.01 wt. % to about 0.05 wt. %, about 0.01 wt. % to about 0.1 wt. %, about 1 wt. % to about 5 wt. %, or about 1 wt. % to about 10 wt. %.

Mushroom (e.g., Tremella fuciformis, Silver Mushroom) and Betaine

Mushrooms, in some embodiments, comprise hydrating properties to retain moisture. Tremella fuciformis can inhibit melanin production. Compositions as described herein, in some embodiments, comprise a mushroom (e.g., Tremella fuciformis, silver mushroom). In some embodiments, the Tremella fuciformis extract is derived from an edible mushroom. In some embodiments, Tremella fuciformis extract provides moisture and antioxidant properties.

In some embodiments, the mushroom (e.g., Tremella fuciformis, silver mushroom) is provided at least or about 0.001 wt. %, 0.005 wt. %, 0.01 wt. %, 0.02 wt. %, 0.05 wt. %, 0.10 wt. %, 0.20 wt. %, 0.25 wt. %, 0.50 wt. %, 0.75 wt. %, 1.0 wt. %, 1.5 wt. %, 2.0 wt. %, 2.5 wt. %, 3.0 wt. %, 3.5 wt. %, 4.0 wt. %, or more than 4 wt. %. In some embodiments, the mushroom (e.g., Tremella fuciformis, silver mushroom) is provided in a range of about 0.25 wt. % to about 10 wt. %, about 0.1 wt. % to about 2.5 wt. %, about 0.5 wt. % to about 8 wt. %, about 0.75 wt. % to about 6 wt. %, or about 1 wt. % to about 4 wt. %. In some embodiments, the mushroom (e.g., Tremella fuciformis, silver mushroom) is provided in a range of about 0.001 wt. % to about 6 wt. %, about 0.002 wt. % to about 4 wt. %, about 0.01 wt. % to about 3 wt. %, or about 0.02 wt. % to about 2 wt. %. In some embodiments the mushroom (e.g., Tremella fuciformis, silver mushroom) is provided at 0.50 wt. %.

In some embodiments, Tremella fuciformis extract is provided at least or about 0.001 wt. %, 0.005 wt. %, 0.01 wt. %, 0.02 wt. %, 0.05 wt. %, 0.10 wt. %, 0.20 wt. %, 0.25 wt. %, 0.50 wt. %, 0.75 wt. %, 1.0 wt. %, 1.5 wt. %, 2.0 wt. %, 2.5 wt. %, 3.0 wt. %, 3.5 wt. %, 4.0 wt. %, or more than 4 wt. %. In some embodiments, the Tremella fuciformis extract is provided in a range of about 0.25 wt. % to about 10 wt. %, about 0.1 wt. % to about 2.5 wt. %, about 0.5 wt. % to about 8 wt. %, about 0.75 wt. % to about 6 wt. %, or about 1 wt. % to about 4 wt. %. In some embodiments, the Tremella fuciformis extract is provided in a range of about 0.001 wt. % to about 6 wt. %, about 0.002 wt. % to about 4 wt. %, about 0.01 wt. % to about 3 wt. %, or about 0.02 wt. % to about 2 wt. %. In some embodiments, the Tremella fuciformis extract is provided at 0.50 wt. %.

Composition as described herein, in some embodiments, comprise betaine. In some embodiments, betaine is provided in provided at least or about 0.001 wt. %, 0.005 wt. %, 0.01 wt. %, 0.02 wt. %, 0.05 wt. %, 0.10 wt. %, 0.20 wt. %, 0.25 wt. %, 0.50 wt. %, 0.75 wt. %, 1.0 wt. %, 1.5 wt. %, 2.0 wt. %, 2.5 wt. %, 3.0 wt. %, 3.5 wt. %, 4.0 wt. %, or more than 4 wt. %. In some embodiments, the betaine is provided in a range of about 0.25 wt. % to about 10 wt. %, about 0.1 wt. % to about 2.5 wt. %, about 0.5 wt. % to about 8 wt. %, about 0.75 wt. % to about 6 wt. %, or about 1 wt. % to about 4 wt. %. In some embodiments, the betaine is provided in a range of about 0.001 wt. % to about 6 wt. %, about 0.002 wt. % to about 4 wt. %, about 0.01 wt. % to about 3 wt. %, or about 0.02 wt. % to about 2 wt. %. In some embodiments, the betaine is provided at 0.50 wt. %.

Composition as described herein, in some embodiments, comprise Tremella fuciformis extract and betaine. In some embodiments, the Tremella fuciformis extract and betaine are provided in provided at least or about 0.001 wt. %, 0.005 wt. %, 0.01 wt. %, 0.02 wt. %, 0.05 wt. %, 0.10 wt. %, 0.20 wt. %, 0.25 wt. %, 0.50 wt. %, 0.75 wt. %, 1.0 wt. %, 1.5 wt. %, 2.0 wt. %, 2.5 wt. %, 3.0 wt. %, 3.5 wt. %, 4.0 wt. %, or more than 4 wt. %. In some embodiments, the Tremella fuciformis extract and betaine are provided in a range of about 0.25 wt. % to about 10 wt. %, about 0.1 wt. % to about 2.5 wt. %, about 0.5 wt. % to about 8 wt. %, about 0.75 wt. % to about 6 wt. %, or about 1 wt. % to about 4 wt. %. In some embodiments, the Tremella fuciformis extract and betaine are provided in a range of about 0.001 wt. % to about 6 wt. %, about 0.002 wt. % to about 4 wt. %, about 0.01 wt. % to about 3 wt. %, or about 0.02 wt. % to about 2 wt. %. In some embodiments, the Tremella fuciformis extract and betaine is provided at 0.50 wt. %.

Hydroxymethoxyphenyl Decanone

Compositions as described herein, in some embodiments, comprise hydroxymethoxyphenyl decanone. Hydroxymethoxyphenyl decanone is a potent intrinsic hyaluronic acid booster, antioxidant and anti-irritant.

In some embodiments, the hydroxymethoxyphenyl decanone is provided in a concentration is provided in a concentration of at least about 0.05 wt. %, 0.1 wt. %, 0.15 wt. %, 0.2 wt. %, 0.25 wt. %, 0.3 wt. %, 0.35 wt. %, 0.4 wt. %, 0.45 wt. %, 0.50 wt. %, or more than 0.50 wt. %. In some embodiments, the hydroxymethoxyphenyl decanone is provided in a range of about 0.25 wt. % to about 10 wt. %, about 0.1 wt. % to about 2.5 wt. %, about 0.5 wt. % to about 8 wt. %, about 0.75 wt. % to about 6 wt. %, or about 1 wt. % to about 4 wt. %. In some embodiments, the hydroxymethoxyphenyl decanone is provided in a range of about 0.001 wt. % to about 6 wt. %, about 0.002 wt. % to about 4 wt. %, about 0.01 wt. % to about 3 wt. %, about 0.01 wt. % to about 3 wt. %, or about 0.02 wt. % to about 2 wt. %. In some embodiments, the hydroxymethoxyphenyl decanone is provided in a concentration of about 0.5 wt. %.

Centella asiatica

Compositions as described herein, in some embodiments, comprise Centella asiatica. With respect to Centella asiatica, it is effective in improving treatment of small wounds, hypertrophic wounds as well as burns, psoriasis and scleroderma. The mechanism of action involves promoting fibroblast proliferation and increasing the synthesis of collagen and intracellular fibronectin content and also improvement of the tensile strength of newly formed skin as well as inhibiting the inflammatory phase of hypertrophic scars and keloids. Research results indicate that it can be used in the treatment of photoaging skin, cellulite and striae. Bylka W, Znajdek-Awizen P, Studzinska-Sroka E, Brzezinska M. Centella asiatica in cosmetology. Postepy Dermatol Alergol 2013; 30(1): 46-9.

In some embodiments, formulations as described herein comprise Centella asiatica. In some embodiments, the Centella asiatica is provided at least or about 0.05 wt. %, 0.10 wt. %, 0.25 wt. %, 0.50 wt. %, 0.75 wt. %, 1.0 wt. %, 1.5 wt. %, 2.0 wt. %, 2.5 wt. %, 3.0 wt. %, 3.5 wt. %, 4.0 wt. %, 4.5 wt. %, 5.0 wt. %, 5.5 wt. %, 6.0 wt. %, 6.5 wt. %, 7.0 wt. %, 8 wt. %, 9 wt. %, 10 wt. %, or more than 10 wt. %. In some embodiments, the Centella asiatica is provided in a range of about 0.25 wt. % to about 10 wt. %, about 0.25 wt. % to about 5 wt. %, about 0.5 wt. % to about 8 wt. %, about 0.75 wt. % to about 6 wt. %, or about 1 wt. % to about 4 wt. %. In some embodiments, the Centella asiatica is provided at about 1.0 wt. %.

Ledum palustre

Compositions as described herein, in some embodiments, comprise Ledum palustre extract. Ledum palustre is an herb that, in some situations, may be referred to as rosemary. In some embodiments, the Ledum palustre is provided in a concentration of at least about 0.05 wt. %, 0.1 wt. %, 0.15 wt. %, 0.2 wt. %, 0.25 wt. %, 0.3 wt. %, 0.35 wt. %, 0.4 wt. %, 0.45 wt. %, 0.50 wt. %, or more than 0.50 wt. %. In some embodiments, the phosphatidylserine is provided in a concentration of about 0.01 wt. % to about 1.0 wt. %, about 0.02 wt. % to about 0.80 wt. %, about 0.03 wt. % to about 0.70 wt. %, about 0.04 wt. % to about 0.60 wt. %, about 0.05 wt. % to about 0.5 wt. %. In some embodiments, the Ledum palustre is provided in a concentration of about 0.1 wt. % to about 2.5 wt. %. In some embodiments, the Ledum palustre is provided in a concentration of about 0.5 wt. %.

Phosphatidylserine

In some embodiments, phospholipids such as phosphatidylserine (PS), a highly enriched membrane phospholipid component, may be added. Phosphatidylserine has been known to have several physiological roles, such as activating signaling enzymes and antioxidant activity (see e.g. Draelos, Z., Pugliese, P. Glycation and Skin Aging: A Review. Cosmetics & Toiletries Magazine 2011; June 2011: 1-6; Lee, S., Yang, J., Park Y., et al. Protective effect and mechanism of phosphatidylserine in UVB-induced human dermal fibroblasts. European Journal of Lipid Science and Technology 2013; 115(7): 783-90; He, M., Kubo, H., Morimoto, K., et al. Receptor for advanced glycation end products binds to phosphatidylserine and assists in the clearance of apoptotic cells. EMBO reports 2011; 12(4): 358-64). It has been found to decrease MMP-1 in a dose dependent manner, to increase procollagen formation and may act as a substrate for AGE targets thus reducing the damage from glycation effects. Clearance of apoptotic cells is necessary for tissue development, homeostasis, and resolution of inflammation. Phosphatidylserine provides an “eat me” signal on the cell surface, and phagocytes recognize the signal using specific receptors such as the receptor of advanced glycation end-products (RAGE). This then binds to PS and assists in the clearance of apoptotic cells and end products of AGE.

Compositions as described herein, in some embodiments, comprise phosphatidylserine. In some embodiments, the phosphatidylserine is provided in a concentration of at least about 0.01 wt. %, 0.02 wt. %, 0.03 wt. %, 0.04 wt. %, 0.05 wt. %, 0.06 wt. %, 0.07 wt. %, 0.08 wt. %, 0.09 wt. %, 0.10 wt. %, 0.11 wt. %, 0.12 wt. % 0.13 wt. %, 0.14 wt. %, 0.15 wt. %, 0.16 wt. %, 0.17 wt. % 0.18 wt. % 0.19 wt. %, 0.20 wt. %, or more than 0.20 wt. %. In some embodiments, the phosphatidylserine is provided in a concentration of about 0.01 wt. % to about 0.20 wt. %, about 0.02 wt. % to about 0.20 wt. %, about 0.03 wt. % to about 0.20 wt. %, about 0.04 wt. % to about 0.20 wt. %, about 0.05 wt. % to about 0.15 wt. %, about 0.06 wt. % to about 0.15 wt. %, about 0.07 wt. % to about 0.15 wt. %, or about 0.08 wt. % to about 0.12 wt. %. In some embodiments, the phosphatidylserine is provided in a concentration of about 0.01 wt. % to about 0.50 wt. %. In some embodiments, the phosphatidylserine is provided in a concentration of about 0.1 wt. %. In some embodiments, the phosphatidylserine is provided in a concentration of at least about 100, 200, 300, 400, 500, 600, 700, 800, 900, 1000, 1100, 1200, 1300, 1400, 1500, 1600, 1700, 1800, 1900, 2000, or more than 2000 parts per million (ppm). In some embodiments, the phosphatidylserine is provided in a concentration of about 100 to about 1900 ppm, about 200 to about 1800 ppm, about 300 to about 1700 ppm, about 400 to about 1600 ppm, about 500 to about 1500 ppm, about 600 to about 1400 ppm, about 700 to about 1300 ppm, about 800 to about 1200 ppm, or about 900 to about 1100 ppm. In some embodiments, the phosphatidylserine is provided in a concentration of about 1000 ppm. In some embodiments, the phosphatidylserine is provided in a concentration of at least about 100, 200, 300, 400, 500, 600, 700, 800, 900, 1000, 1100, 1200, 1300, 1400, 1500, 1600, 1700, 1800, 1900, 2000, or more than 2000 μg/ml In some embodiments, the phosphatidylserine is provided in a concentration of about 100 to about 1900 μg/ml, about 200 to about 1800 μg/ml, about 300 to about 1700 μg/ml, about 400 to about 1600 μg/ml, about 500 to about 1500 μg/ml, about 600 to about 1400 μg/ml, about 700 to about 1300 μg/ml, about 800 to about 1200 μg/ml, or about 900 to about 1100 μg/ml. In some embodiments, the phosphatidylserine is provided in a concentration of about 500 to about 1000 μg/ml.

Arnica montana Extract

Arnica montana extract includes components such as essential oils, fatty acids, thymol, pseudoguaianolide sesquiterpene lactones and flavanone glycosides. It can exhibit an anti-inflammatory effect.

In some embodiments, Arnica montana extract is provided at least or about 0.001 wt. %, 0.005 wt. %, 0.01 wt. %, 0.02 wt. %, 0.05 wt. %, 0.10 wt. %, 0.20 wt. %, 0.25 wt. %, 0.50 wt. %, 0.75 wt. %, 1.0 wt. %, 1.5 wt. %, 2.0 wt. %, 2.5 wt. %, 3.0 wt. %, 3.5 wt. %, 4.0 wt. %, or more than 4 wt. %. In some embodiments, the Arnica montana extract is provided in a range of about 0.25 wt. % to about 10 wt. %, about 0.1 wt. % to about 2.5 wt. %, about 0.5 wt. % to about 8 wt. %, about 0.75 wt. % to about 6 wt. %, or about 1 wt. % to about 4 wt. %. In some embodiments, the Arnica montana extract is provided in a range of about 0.001 wt. % to about 6 wt. %, about 0.002 wt. % to about 4 wt. %, about 0.01 wt. % to about 3 wt. %, or about 0.02 wt. % to about 2 wt. wt. %. In some embodiments, the Arnica montana extract is provided at 0.50 wt. %.

Phytoene and Phytofluene

Compositions as described herein, in some embodiments, comprise phytoene, phytofluene, or combinations thereof. Phytoene and phytofluene are colorless carotenoids derived from saltwater microalgae that modulate Prostaglandin E-2 (PGE-2).

In some embodiments, the phytoene, phytofluene, or combinations thereof is provided at least or about 0.001%, 0.005%, 0.01%, 0.02%, 0.05%, 0.10%, 0.20%, 0.25%, 0.50%, 0.75%, 1.0%, 1.5%, 2.0%, 2.5%, 3.0%, 3.5%, 4.0%, or more than 4% by weight (wt. %). In some embodiments, the phytoene, phytofluene, or combinations thereof is provided in a range of about 0.25% to about 10%, about 0.1% to about 2.5%, about 0.5% to about 8%, about 0.75% to about 6%, or about 1% to about 4% by weight. In some embodiments, the phytoene, phytofluene, or combinations thereof is provided in a range of about 0.001% to about 6%, about 0.002% to about 4%, about 0.01% to about 3%, or about 0.02% to about 2% by weight.

Xylitylglucoside, Anhydroxylitol, and Xylitol

In some embodiments, compositions described herein comprise xylitylglucoside. In some embodiments, the xylitylglucoside is provided at least or about 0.05 wt. %, 0.10 wt. %, 0.25 wt. %, 0.50 wt. %, 0.75 wt. %, 1.0 wt. %, 1.5 wt. %, 2.0 wt. %, 2.5 wt. %, 3.0 wt. %, 3.5 wt. %, 4.0 wt. %, 4.5 wt. %, 5.0 wt. %, 5.5 wt. %, 6.0 wt. %, 6.5 wt. %, 7.0 wt. %, 8 wt. %, 9 wt. %, 10 wt. %, or more than 10 wt. %. In some embodiments, the xylitylglucoside is provided in a range of about 0.25 wt. % to about 10 wt. %, about 0.5 wt. % to about 8 wt. %, about 0.75 wt. % to about 6 wt. %, or about 1 wt. % to about 4 wt. wt. %. In some embodiments, the xylitylglucoside is provided at about 1.0 wt. %.

In some embodiments, compositions described herein comprise anhydroxylitol. In some embodiments, the anhydroxylitol is provided at least or about 0.05 wt. %, 0.10 wt. %, 0.25 wt. %, 0.50 wt. %, 0.75 wt. %, 1.0 wt. %, 1.5 wt. %, 2.0 wt. %, 2.5 wt. %, 3.0 wt. %, 3.5 wt. %, 4.0 wt. %, 4.5 wt. %, 5.0 wt. %, 5.5 wt. %, 6.0 wt. %, 6.5 wt. %, 7.0 wt. %, 8 wt. %, 9 wt %, 10 wt. % or more than 10 wt. %. In some embodiments, the anhydroxylitol is provided in a range of about 0.25 wt. % to about 10 wt. %, about 0.5 wt. % to about 8 wt. %, about 0.75 wt. % to about 6 wt. %, or about 1 wt. % to about 4 wt. %. In some embodiments, the anhydroxylitol is provided at about 1.0 wt. %.

In some embodiments, compositions described herein comprise xylitol. In some embodiments, the xylitol is provided at least or about 0.05 wt. %, 0.10 wt. %, 0.25 wt. %, 0.50 wt. %, 0.75 wt. %, 1.0 wt. %, 1.5 wt. %, 2.0 wt. %, 2.5 wt. %, 3.0 wt. %, 3.5 wt. %, 4.0 wt. %, 4.5 wt. %, 5.0 wt. %, 5.5 wt. %, 6.0 wt. %, 6.5 wt. %, 7.0 wt. %, 8 wt. %, 9 wt. %, 10 wt. %, or more than 10 wt. %. In some embodiments, the xylitol is provided in a range of about 0.25 wt. % to about 10 wt. %, about 0.5 wt. % to about 8 wt. %, about 0.75 wt. % to about 6 wt. %, or about 1 wt. % to about 4 wt. %. In some embodiments, the xylitol is provided at about 1.0 wt. %.

In some embodiments, compositions described herein comprise xylitylglucoside, anhydroxylitol, or xylitol. In some embodiments, the xylitylglucoside, anhydroxylitol, or xylitol are provided at least or about 0.05 wt. %, 0.10 wt. %, 0.25 wt. %, 0.50 wt. %, 0.75 wt. %, 1.0 wt. %, 1.5 wt. %, 2.0 wt. %, 2.5 wt. %, 3.0 wt. %, 3.5 wt. %, 4.0 wt. %, 4.5 wt. %, 5.0 wt. %, 5.5 wt. %, 6.0 wt. %, 6.5 wt. %, 7.0 wt. %, 8 wt. % 9 wt. %, 10 wt. %, or more than 10 wt. %. In some embodiments, the xylitylglucoside, anhydroxylitol, or xylitol are provided in a range of about 0.25 wt. % to about 10 wt. %, about 0.5 wt. % to about 8 wt. %, about 0.75 wt. % to about 6 wt. %, or about 1 wt. % to about 4 wt. %. In some embodiments, the xylitylglucoside, anhydroxylitol, or xylitol are provided at about 1.0 wt. %.

In some embodiments, compositions described herein comprise xylitylglucoside, anhydroxylitol, and xylitol. In some embodiments, the xylitylglucoside, anhydroxylitol, and xylitol are provided at least or about 0.05 wt. %, 0.10 wt. %, 0.25 wt. %, 0.50 wt. %, 0.75 wt. %, 1.0 wt. %, 1.5 wt. %, 2.0 wt. %, 2.5 wt. %, 3.0 wt. %, 3.5 wt. %, 4.0 wt. %, 4.5 wt. %, 5.0 wt. %, 5.5 wt. %, 6.0 wt. %, 6.5 wt. %, 7.0 wt. %, 8 wt. %, 9 wt. %, 10 wt. %, or more than 10 wt. %. In some embodiments, the xylitylglucoside, anhydroxylitol, and xylitol are provided in a range of about 0.25 wt. % to about 10 wt. %, about 0.2 wt. % to about 5 wt. %, about 0.5 wt. % to about 8 wt. %, about 0.75 wt. % to about 6 wt. %, or about 1 wt. % to about 4 wt. %. In some embodiments, the xylitylglucoside, anhydroxylitol, and xylitol are provided at about 1.0 wt. %.

Tetrandrine

In some embodiments, compositions described herein comprise tetrandrine. Tetrandrine is a bisbenzylisoquinoline alkaloid derived from S. tetrandra. In some embodiments, tetrandrine modulates inflammation, cell proliferation, collagen synthesis, DNA synthesis, apoptosis, or combinations thereof. In some embodiments, tetrandrine modulates gene expression of genes involved in scar formation. Exemplary genes include, but are not limited to, TGF-β1, TGF-β3, SMAD-2, and SMAD-7. In some embodiments, tetrandrine increases expression of SMAD-7. In some embodiments, tetrandrine decreases expression of SMAD-2.

In some embodiments, tetrandrine is provided at least or about 0.0005 wt. %, 0.00075 wt. %, 0.0010 wt. %, 0.0020 wt. %, 0.0030 wt. %, 0.0040 wt. %, 0.005 wt. %, or more than 0.005 wt. %. In some embodiments, the tetrandrine is provided in a range of about 0.0001 wt. % to about 0.0005 wt. %, about 0.0002 wt. % to about 0.0005 wt. %, about 0.0003 wt. % to about 0.005 wt. %, 0.0005 wt. % to about 0.005 wt. %, or about 0.0005 wt. % to about 0.0025 wt. %. In some embodiments, the tetrandrine is provided at about 0.001 wt. %.

Other Components

Other components can include anti-inflammatory agents, antioxidants, and solubility enhancers. Exemplary anti-irritation agents include, but are not limited to, panthenyl triacetate and naringenin. Panthenyl triacetate and naringenin are natural plant extracts that reduce redness and water loss through the skin. Typical amounts for anti-irritation agents when employed in compositions are from 1 wt. % to 4 wt. %.

Embodiments as disclosed herein, in some features, comprise panthenyl triacetate. In some embodiments, the panthenyl triacetate is provided at least or about 0.001 wt. %, 0.005 wt. %, 0.01 wt. %, 0.02 wt. %, 0.05 wt. %, 0.10 wt. %, 0.20 wt. %, 0.25 wt. %, 0.50 wt. %, 0.75 wt. %, 1.0 wt. %, 1.5 wt. %, 2.0 wt. %, 2.5 wt. %, 3.0 wt. %, 3.5 wt. %, 4.0 wt. %, or more than 4 wt. %. In some embodiments, the panthenyl triacetate is provided in a range of about 0.001 wt. % to about 4.0 wt. %, about 0.01 wt. % to about 3.0 wt. %, about 0.1 wt. % to about 2.5 wt. %, or about 0.50 wt. % to about 1.5 wt. %. In some embodiments, the panthenyl triacetate is provided at about 2.00 wt. %.

Embodiments as disclosed herein, in some features, comprise naringenin. In some embodiments, the naringenin is provided at least or about 0.001 wt. %, 0.005 wt. %, 0.01 wt. %, 0.02 wt. %, 0.05 wt. %, 0.10 wt. %, 0.20 wt. %, 0.25 wt. %, 0.50 wt. %, 0.75 wt. %, 1.0 wt. %, 1.5 wt. %, 2.0 wt. %, 2.5 wt. %, 3.0 wt. %, 3.5 wt. %, 4.0 wt. %, or more than 4 wt. %. In some embodiments, the naringenin is provided in a range of about 0.001 wt. % to about 4.0 wt. %, about 0.01 wt. % to about 3.0 wt. %, about 0.1 wt. % to about 2.5 wt. %, or about 0.50 wt. % to about 1.5 wt. %. In some embodiments, the naringenin is provided at about 2.00 wt. %.

Embodiments as disclosed herein, in some features, comprise panthenyl triacetate or naringenin. In some embodiments, the panthenyl triacetate or naringenin is provided at least or about 0.001 wt. %, 0.005 wt. %, 0.01 wt. %, 0.02 wt. %, 0.05 wt. %, 0.10 wt. %, 0.20 wt. %, 0.25 wt. %, 0.50 wt. %, 0.75 wt. %, 1.0 wt. %, 1.5 wt. %, 2.0 wt. %, 2.5 wt. %, 3.0 wt. %, 3.5 wt. %, 4.0 wt. %, or more than 4 wt. %. In some embodiments, the panthenyl triacetate or naringenin is provided in a range of about 0.001 wt. % to about 4.0 wt. %, about 0.01 wt. % to about 3.0 wt. %, about 0.1 wt. % to about 2.5 wt. %, or about 0.50 wt. % to about 1.5 wt. %. In some embodiments, the panthenyl triacetate or naringenin is provided at least or about 2.00 wt. %.

Embodiments as disclosed herein, in some features, comprise panthenyl triacetate and naringenin. In some embodiments, the panthenyl triacetate and naringenin is provided at least or about 0.001 wt. %, 0.005 wt. %, 0.01 wt. %, 0.02 wt. %, 0.05 wt. %, 0.10 wt. %, 0.20 wt. %, 0.25 wt. %, 0.50 wt. %, 0.75 wt. %, 1.0 wt. %, 1.5 wt. %, 2.0 wt. %, 2.5 wt. %, 3.0 wt. %, 3.5 wt. %, 4.0 wt. %, 5.0 wt. %, 6.0 wt. %, 7.0 wt. %, 8.0 wt. %, 9.0 wt. %, 10.0 wt. %, or more than 10.0 wt. %. In some embodiments, the panthenyl triacetate and naringenin is provided in a range of about 0.001 wt. % to about 4.0 wt. %, about 0.01 wt. % to about 3.0 wt. %, about 0.1 wt. % to about 2.5 wt. %, or about 0.50 wt. % to about 1.5 wt. %. In some embodiments, the panthenyl triacetate and naringenin is provided in a range of about 0.4 wt. % to about 10.0 wt. %. In some embodiments, the panthenyl triacetate and naringenin is provided at least or about 2.00 wt. %.

Exemplary antioxidant agents include, but are not limited to, Dunaliella salina extract and squalane. Dunaliella salina extract includes components such as beta carotenes. It can exhibit an antioxidant effect. Typical amounts for anti-inflammatory agents when employed in compositions are from 0.1 wt. % to 2.5 wt. %.

Embodiments as disclosed herein, in some features, comprise Dunaliella salina extract. In some embodiments, the Dunaliella salina extract is provided at least or about 0.001 wt. %, 0.005 wt. %, 0.01 wt. %, 0.02 wt. %, 0.05 wt. %, 0.10 wt. %, 0.20 wt. %, 0.25 wt. %, 0.50 wt. %, 0.75 wt. %, 1.0 wt. %, 1.5 wt. %, 2.0 wt. %, 2.5 wt. %, 3.0 wt. %, 3.5 wt. %, 4.0 wt. %, or more than 4 wt. %. In some embodiments, the Dunaliella salina extract is provided in a range of about 0.001 wt. % to about 4.0 wt. %, about 0.01 wt. % to about 3.0 wt. %, about 0.1 wt. % to about 2.5 wt. %, or about 0.50 wt. % to about 1.5 wt. % In some embodiments, the Dunaliella salina extract is provided at or about 0.50 wt. %.

Embodiments as disclosed herein, in some features, comprise squalene. In some embodiments, the squalane is provided at least or about 0.001 wt. %, 0.005 wt. %, 0.01 wt. %, 0.02 wt. %, 0.05 wt. %, 0.10 wt. %, 0.20 wt. %, 0.25 wt. %, 0.50 wt. %, 0.75 wt. %, 1.0 wt. %, 1.5 wt. %, 2.0 wt. %, 2.5 wt. %, 3.0 wt. %, 3.5 wt. %, 4.0 wt. %, or more than 4 wt. %. In some embodiments, the squalane is provided in a range of about 0.001 wt. % to about 4.0 wt. %, about 0.01 wt. % to about 3.0 wt. %, about 0.1 wt. % to about 2.5 wt. %, or about 0.50 wt. % to about 1.5 wt. %. In some embodiments, the squalane is provided at or about 0.50 wt. %.

Embodiments as disclosed herein, in some features, comprise Dunaliella salina extract or squalane. In some embodiments, the Dunaliella salina extract or the squalane is provided at least or about 0.001 wt. %, 0.005 wt. %, 0.01 wt. %, 0.02 wt. %, 0.05 wt. %, 0.10 wt. %, 0.20 wt. %, 0.25 wt. %, 0.50 wt. %, 0.75 wt. %, 1.0 wt. %, 1.5 wt. %, 2.0 wt. %, 2.5 wt. %, 3.0 wt. %, 3.5 wt. %, 4.0 wt. %, or more than 4 wt. %. In some embodiments, the Dunaliella salina or the squalane is provided in a range of about 0.001 wt. % to about 4.0 wt. %, about 0.01 wt. % to about 3.0 wt. %, about 0.1 wt. % to about 2.5 wt. %, or about 0.50 wt. % to about 1.5 wt. %. In some embodiments, the Dunaliella salina or the squalane extract is provided at or about 0.50 wt. %.

Embodiments as disclosed herein, in some features, comprise Dunaliella salina extract and squalane. In some embodiments, the Dunaliella salina extract and the squalane is provided at least or about 0.001 wt. %, 0.005 wt. %, 0.01 wt. %, 0.02 wt. %, 0.05 wt. %, 0.10 wt. %, 0.20 wt. %, 0.25 wt. %, 0.50 wt. %, 0.75 wt. %, 1.0 wt. %, 1.5 wt. %, 2.0 wt. %, 2.5 wt. %, 3.0 wt. %, 3.5 wt. %, 4.0 wt. %, or more than 4 wt. %. In some embodiments, the Dunaliella salina and the squalane is provided in a range of about 0.001 wt. % to about 4.0 wt. %, about 0.01 wt. % to about 3.0 wt. %, about 0.1 wt. % to about 2.5 wt. %, or about 0.50 wt. % to about 1.5 wt. %. In some embodiments, the Dunaliella salina and the squalane extract is provided at or about 0.50 wt. %.

In some embodiments, the peptides are in admixture with a suitable carrier, diluent, or excipient, and can contain auxiliary substances such as wetting or emulsifying agents, pH buffering agents, gelling or viscosity enhancing additives, preservatives, scenting agents, colors, and the like, depending upon the route of administration and the preparation desired. See, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively). Such preparations can include complexing agents, metal ions, polymeric compounds such as polyacetic acid, polyglycolic acid, hydrogels, dextran, and the like, liposomes, microemulsions, micelles, unilamellar or multilamellar vesicles, erythrocyte ghosts or spheroblasts. Suitable lipids for compositions include, without limitation, monoglycerides, diglycerides, sulfatides, lysolecithin, phospholipids, saponin, bile acids, and the like. In some embodiments, compositions described herein comprise, phosphatidylserine, phospholipids, tocopherol, ascorbyl palmitate, or combinations thereof. In some embodiments, phosphatidylserine, phospholipids, tocopherol, ascorbyl palmitate, or combinations thereof is provided at 0.001 wt. %, 0.005 wt. %, 0.01 wt. %, 0.02 wt. %, 0.05 wt. %, 0.10 wt. %, 0.20 wt. %, 0.25 wt. %, 0.50 wt. %, 0.75 wt. %, 1.0 wt. %, 1.5 wt. %, 2.0 wt. %, 2.5 wt. %, 3.0 wt. %, 3.5 wt. %, 4.0 wt. %, or more than 4 wt. %. In some embodiments, the phosphatidylserine, phospholipids, tocopherol, ascorbyl palmitate, or combinations thereof is provided in a range of about 0.25 wt. % to about 10 wt. %, about 0.5 wt. % to about 8 wt. %, about 0.75 wt. % to about 6 wt. %, or about 1 wt. % to about 4 wt. %. In some embodiments, the phosphatidylserine, phospholipids, tocopherol, ascorbyl palmitate, or combinations thereof is provided in a range of about 0.001 wt. % to about 6 wt. %, about 0.002 wt. % to about 4 wt. %, about 0.01 wt. % to about 3 wt. %, or about 0.02 wt. % to about 5 wt. %. In some embodiments, the additive is betaine. Betaine, in some embodiments, is provided in a range of about 0.001 wt. % to about 6 wt. %, about 0.002 wt. % to about 4 wt. %, about 0.01 wt. % to about 3 wt. %, or about 0.02 wt. % to about 5 wt. %. In some embodiments, the compositions as described herein comprise caprylyl glycol. In some embodiments, the caprylyl glycol provided in a range of about 0.001 wt. % to about 6 wt. %, about 0.002 wt. % to about 4 wt. %, about 0.01 wt. % to about 3 wt. %, or about 0.02 wt. % to about 5 wt. %. In some embodiments, the compositions as described herein comprise caprylhydroxamic acid. In some embodiments, the caprylhydroxamic acid provided in a range of about 0.001 wt. % to about 6 wt. %, about 0.002 wt. % to about 4 wt. %, about 0.01 wt. % to about 3 wt. %, or about 0.02 wt. % to about 5 wt. %. The presence of such additional components can influence the physical state, solubility, stability, rate of release, rate of clearance, and penetration of active ingredients.

The compositions for topical administration comprise the peptide compositions as described herein and a dermatologically acceptable vehicle. The vehicle may be aqueous or nonaqueous. The dermatologically acceptable vehicle used in the topical composition may be in the form of a lotion, a gel, an ointment, a liquid, a cream, or an emulsion. If the vehicle is an emulsion, the emulsion may have a continuous aqueous phase and a discontinuous nonaqueous or oil phase (oil-in-water emulsion), or a continuous nonaqueous or oil phase and a discontinuous aqueous phase (water-in-oil emulsion). When administered topically in liquid or gel form, a liquid carrier such as water, petroleum, oils of animal or plant origin such as peanut oil, mineral oil, soybean oil, or sesame oil, or synthetic oils can be added to the active ingredient(s). Physiological saline solution, dextrose, or other saccharide solution, or glycols such as ethylene glycol, propylene glycol, or polyethylene glycol are also suitable liquid carriers. The pharmaceutical compositions can also be in the form of oil-in-water emulsions. The oily phase can be a vegetable oil, such as olive or arachis oil, a mineral oil such as liquid paraffin, or a mixture thereof. Suitable emulsifying agents include naturally-occurring gums such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsions can also contain coloring and scenting agents.

In some embodiments, a silicone elastomer (e.g., dimethicone crosspolymer) is employed to increase delivery and penetration of the peptides into the skin. An alternative to increasing molecular weight (as with silicone gums) or adding filler (as with silicone compounds) is to partially crosslink siloxane polymers and disperse this material in an appropriate silicone carrier fluid. The resulting dimethicone crosspolymers (also known as silicone elastomers in the personal care industry) differ from basic polydimethylsiloxane (PDMS) because of the cross-linking between the linear polymers. These materials can be employed in peptide compositions, and also offer benefits in scar treatment, periwound protection and enzyme delivery. In skin care applications, the aesthetics of silicone elastomers (including those with functional groups) and their ability to absorb various oils (e.g., with a dimethicone/vinyl dimethicone crosspolymer such as Dow Corning® 9506 Elastomer Powder) are two of the elastomer's desirable properties. Silicone elastomers have a skin feel different from any of the silicone fluids, described as “smooth,” “velvety,” and “powdery.” It can be modified by controlling the amount of liquid phase in the formula, and therefore the degree of swelling. Due to their film-forming properties, dimethicone crosspolymers can be used as delivery systems for active ingredients such as the peptides described herein, or other composition components such as oil-soluble vitamins and sunscreens. Sunscreens such as octyl methoxycinnamate can be more efficiently delivered from a composition containing a silicone elastomer, producing a higher sun protection factor (SPF). Silicone elastomer blends can be used to enhance SPF in oil-in-water compositions containing organic sunscreens. For example, in testing conducted regarding SPF, the addition of 4% silicone elastomer blend to a sun care composition containing organic sunscreens increased the SPF from 5.7 to 18. This property of the silicone elastomer allows the effectiveness of sunscreen agents in a composition to be maximized while reducing the amount needed to achieve a desired SPF. As a result, composition costs can be reduced along with potential irritation caused by sunscreen actives. Accordingly, a higher SPF can be achieved with the same amount of UV absorber, resulting in enhanced performance with no added composition cost. Silicone elastomers can be produced from linear silicone polymers by a variety of crosslinking reactions, e.g., by a hydrosilylation reaction in which a vinyl group reacts with a silicon hydride. The general process involves linear silicone polymers with reactive sites along the polymer chain reacting with a cross-linker. The dimethicone crosspolymer can be produced either as a gel made of a suspension of elastomer particles swollen in a carrier fluid (e.g., a mixture of high molecular weight silicone elastomer in cyclopentasiloxane such as Dow Corning® 9040 Silicone Elastomer Blend), or as a spray-dried powder (a dimethicone/vinyl dimethicone crosspolymer such as Dow Corning® 9506 Elastomer Powder). The gel form having desirable attributes is cyclomethicone, but low viscosity dimethicones and organic fluids can also be used. Examples of dimethicone crosspolymers in the suspension or gel form are high molecular weight silicone elastomer (12%) in decamethylcyclopentasiloxane (e.g., Dow Corning® ST-Elastomer 10) and a mixture of high molecular weight silicone elastomer in cyclopentasiloxane (e.g., Dow Corning® 9040 Silicone Elastomer Blend), which typically have an elastomer content ranging from 10 wt. % to 20 wt. %.

In some embodiments, the composition comprises a siloxane polymer. In some embodiments, the siloxane polymer is caprylyl methicone. In some embodiments, caprylyl methicone is provided at least or about 0.001 wt. %, 0.005 wt. %, 0.01 wt. %, 0.02 wt. %, 0.05 wt. %, 0.10 wt. %, 0.20 wt. %, 0.25 wt. %, 0.50 wt. %, 0.75 wt. %, 1.0 wt. %, 1.5 wt. %, 2.0 wt. %, 2.5 wt. %, 3.0 wt. %, 3.5 wt. %, 4.0 wt. %, or more than 4.0 wt. %. In some embodiments, the caprylyl methicone is provided at about 0.5 wt. %. In some embodiments, the caprylyl methicone is provided in a range of about 0.001 wt. % to about 4.0 wt. %, about 0.01 wt. % to about 3.0 wt. %, about 0.1 wt. % to about 2.5 wt. %, or about 0.50 wt. % to about 1.5 wt. %. In some embodiments, the caprylyl methicone is provided at about 0.25 wt. %. In some embodiments, the caprylyl methicone is provided at about 1 wt. %.

Bentonite clays can be employed in conjunction with the peptides to provide impart penetration and adsorption properties to the compositions, and can aid in stabilizing emulsions. Other clays, such as hectorite and magnesium aluminum silicate can also be employed. Bentonite or other clays can be modified to yield an organic modified clay compound. Salts (e.g., quaternary ammonium salts) of fatty acids (e.g., hydrogenated fatty acids) can be reacted with hectorite or other clays. As provided herein, fatty acids are referred to and described using conventional nomenclature as is employed by one of skill in the art. A saturated fatty acid includes no carbon-carbon double bonds. An unsaturated fatty acid includes at least one carbon-carbon double bond. A monounsaturated fatty acid includes only one carbon-carbon double bond. A polyunsaturated fatty acid includes two or more carbon-carbon double bonds. Double bonds in fatty acids are generally cis; however, trans double bonds are also possible. The position of double bonds can be indicated by Δn, where n indicates the lower numbered carbon of each pair of double-bonded carbon atoms. A shorthand notation specifying total #carbons:#double bonds, Δ_{double bond positions} can be employed. For example, 20:4Δ_{5,8,11,14} refers to a fatty acid having 20 carbon atoms and four double bonds, with the double bonds situated between the 5 and 6 carbon atom, the 8 and 9 carbon atom, the 11 and 12 carbon atom, and the 14 and 15 carbon atom, with carbon atom 1 being the carbon of the carboxylic acid group. Stearate (octadecanoate) is a saturated fatty acid. Oleate (cis-Δ9-octadecenoate) is a monounsaturated fatty acid, linolenate (all-cis-Δ9,12,15-octadecatrienoate) is a polyunsaturated fatty acid. Fatty acids suitable for use can comprise from 5 to 30 carbon atoms, e.g., 5, 6, 7, 8, 9, 10, 11, 12, 13, 14, 15, 16, 17, 18, 19, 20, 21, 22, 23, 24, 25, 26, 27, 28, 29, or 30 carbon atoms. The fatty acid can be fully saturated, or can include as many double bonds as are feasible for the chain length. Fatty acids suitable for functionalizing hectorite or other clays include palmitic acid and stearic acid. Dialkyl quaternary cationic modifiers include dipalmoyldimonium chloride and distearyldimonium chloride. Amidoamine quaternary cationic modifiers include palmitamidopropyltrimonium chloride cetearyl alcohol and palmitamidopropyltrimonium chloride.

The pharmaceutical excipients used in the topical preparations of the peptide compositions may be selected from the group consisting of solvents, emollients and/or emulsifiers, oil bases, preservatives, antioxidants, tonicity adjusters, penetration enhancers and solubilizers, chelating agents, buffering agents, surfactants, one or more polymers, and combinations thereof.

Suitable solvents for an aqueous or hydrophilic liposomal composition include water; ethyl alcohol; isopropyl alcohol; mixtures of water and ethyl and/or isopropyl alcohols; glycerin; ethylene, propylene or butylene glycols; DMSO; and mixtures thereof. In some embodiments, glycerin is provided at least or about 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, or more than 12 wt. %. In some embodiments, glycerin is provided at least or about 7 wt. %. In some embodiments, glycerin is provided in a range of about 1 wt. % to about 12 wt. %, about 2 wt. % to about 11 wt. %, or about 3 wt. % to about 10 wt. %. In some embodiments, butylene glycol is provided at least or about 0.0025 wt. %, 0.005 wt. %, 0.075 wt. %, 0.01 wt. %, 0.025 wt. %, 0.05 wt. %, 0.75 wt. %, 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, or more than 12 wt. %. In some embodiments, butylene glycol is provided in a range of about 0.01 wt. % to about 10 wt. %, about 0.025 wt. % to about 5 wt. %, or about 0.05 wt. % to about 1.25 wt. %. Suitable solvents for hydrophobic compositions include mineral oils, vegetable oils, and silicone oils. If desired, the peptide compositions as described herein may be dissolved or dispersed in a hydrophobic oil phase, and the oil phase may then be emulsified in an aqueous phase comprising water, alone or in combination with lower alcohols, glycerin, and/or glycols. In some embodiments, an anhydrous composition is applied as the presence of water can result in stinging upon administration to skin tissues subject to laser treatment, chemical peel, dermabrasion, or the like. Anhydrous compositions may also act to prevent the development of water-based irritant contact dermatitis in damaged or sensitive skin, which may produce rashes and skin irritation that may retard wound healing and improvement in skin quality. Tsai, T. F., Maibach, H. I. How irritant is water? An overview. Contact Dermatitis 41(6) (1999): 311-314 (describing contact dermatitis caused by water as an irritant). However, in some embodiments it may be acceptable to provide water-based compositions, or to permit a limited amount of water to be present. For example, water may be present, but at amounts below the threshold at which a stinging sensation when applied to damaged skin may result. Osmotic shock or osmotic stress is a sudden change in the solute concentration around a cell, causing a rapid change in the movement of water across its cell membrane. Under conditions of high concentrations of either salts, substrates or any solute in the supernatant, water is drawn out of the cells through osmosis. This also inhibits the transport of substrates and cofactors into the cell thus “shocking” the cell. Alternatively, at low concentrations of solutes, water enters the cell in large amounts, causing it to swell and either burst or undergo apoptosis. Certain of the compositions as described herein can be advantageously employed where it is desirable to minimize osmotic shock.

Compositions as described herein may comprise varying amounts of solvent. In some embodiments, the solvent is water. In some embodiments, the solvent is at least or about 10 wt. %, 15 wt. %, 20 wt. %, 25 wt. %, 30 wt. %, 35 wt. %, 40 wt. %, 45 wt. %, 50 wt. %, 55 wt. %, 60 wt. %, 65 wt. %, 70 wt. %, 75 wt. %, 80 wt. %, 85 wt. %, 90 wt. %, 95 wt. %, or more than 95 wt. %. In some embodiments, the solvent is in a range of about 10 wt. % to about 95 wt. %, about 20 wt. % to about 90 wt. %, about 30 wt. % to about 85 wt. %, about 40 wt. % to about 80 wt. %, or about 50 wt. % to about 75 wt. %.

Viscosity of the compositions can be maintained at the selected level using a pharmaceutically acceptable thickening agent. Suitable viscosity enhancers or thickeners which may be used to prepare a viscous gel or cream with an aqueous base include sodium polyacrylate, xanthan gum, polyvinyl pyrrolidone, acrylic acid polymer, carrageenans, hydroxyethyl cellulose, hydroxypropyl cellulose, methyl cellulose, ethyl cellulose, propyl cellulose, hydroxypropyl methyl cellulose, polyethoxylated polyacrylamides, polyethoxylated acrylates, and polyethoxylated alkane thiols. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the thickening agent selected. An amount is preferably used that will achieve the selected viscosity. Viscous compositions are normally prepared from solutions by the addition of such thickening agents, or by employing a base that has an acceptable level of viscosity.

The viscosity of the compositions as described herein, in some embodiments, are in a range of about 8,000 centipoise (cps) to about 30,000 cps. In some embodiments, the viscosity is at least or about 4,000; 5,000; 6,000; 7,000; 8,000; 9,000; 10,000; 11,000; 12,000; 13,000; 14,000; 15,000; 16,000; 17,000; 18,000; 19,000; 20,000; 21,000; 22,000; 23,000; 24,000; 25,000; 26,000; 27,000; 28,000; 29,000; 30,000; 31,000; 32,000; 33,000; 34,000, 35,000; 36,000; 37,000; 38,000; 39,000; 40,000; or more than 40,000 cps. In some embodiments, the composition comprises a viscosity in a range of about 4,000 to about 40,000, about 6,000 to about 38,000, about 8,000 to about 36,000, about 10,000 to about 34,000 cps, about 12,000 to about 32,000 cps, or about 14,000 to about 30,000 cps.

Suitable emollients include hydrocarbon oils and waxes such as mineral oil, petrolatum, paraffin, ceresin, ozokerite, microcrystalline wax, polyethylene, squalene, perhydrosqualene, silicone oils, triglyceride esters, acetoglyceride esters, such as acetylated monoglycerides; ethoxylated glycerides, such as ethoxylated glyceryl monostearate; alkyl esters of fatty acids or dicarboxylic acids. In some embodiments, the emollient is caprylic/capric triglyceride.

In some embodiments, the emollient is provided at least or about 0.0025 wt. %, 0.005 wt. %, 0.075 wt. %, 0.01 wt. %, 0.025 wt. %, 0.05 wt. %, 0.75 wt. %, 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, or more than 12 wt. %. In some embodiments, the emollient is provided in a range of about 0.01 wt. % to about 10 wt. %, about 0.01 wt. % to about 2.5 wt. %, about 0.025 wt. % to about 5 wt. %, or about 0.05 wt. % to about 1.25 wt. %. In some embodiments, the caprylic/capric triglyceride is provided at least or about 0.0025 wt. %, 0.005 wt. %, 0.075 wt. %, 0.01 wt. %, 0.025 wt. %, 0.05 wt. %, 0.75 wt. %, 1 wt. %, 2 wt. %, 3 wt. %, 4 wt. %, 5 wt. %, 6 wt. %, 7 wt. %, 8 wt. %, 9 wt. %, 10 wt. %, 11 wt. %, 12 wt. %, or more than 12 wt. %. In some embodiments, the caprylic/capric triglyceride is provided in a range of about 0.01 wt. % to about 10 wt. %, about 0.01 wt. % to about 2.5 wt. %, about 0.025 wt. % to about 5 wt. %, or about 0.05 wt. % to about 1.25 wt. %.

Suitable silicone oils for use as emollients include dimethyl polysiloxanes, methyl(phenyl) polysiloxanes, and water-soluble and alcohol-soluble silicone glycol copolymers. Suitable triglyceride esters for use as emollients include vegetable and animal fats and oils including castor oil, safflower oil, cotton seed oil, corn oil, olive oil, cod liver oil, almond oil, avocado oil, palm oil, sesame oil, and soybean oil.

Suitable esters of carboxylic acids or diacids for use as emollients include methyl, isopropyl, and butyl esters of fatty acids. Specific examples of alkyl esters including hexyl laurate, isohexyl laurate, iso-hexyl palmitate, isopropyl palmitate, decyl oleate, isodecyl oleate, hexadecyl stearate, decyl stearate, isopropyl isostearate, dilauryl lactate, myristyl lactate, and cetyl lactate; and alkenyl esters of fatty acids such as oleyl myristate, oleyl stearate, and oleyl oleate. Specific examples of alkyl esters of diacids include diisopropyl adipate, diisohexyl adipate, bis(hexyldecyl) adipate, and diisopropyl sebacate.

Other suitable classes of emollients or emulsifiers which may be used in the compositions include fatty acids, fatty alcohols, fatty alcohol ethers, ethoxylated fatty alcohols, fatty acid esters of ethoxylated fatty alcohols, and waxes.

Specific examples of fatty acids for use as emollients include pelargonic, lauric, myristic, palmitic, stearic, isostearic, hydroxystearic, oleic, linoleic, ricinoleic, arachidic, behenic, and erucic acids. Specific examples of fatty alcohols for use as emollients include lauryl, myristyl, cetyl, hexadecyl, stearyl, isostearyl, hydroxystearyl, oleyl, ricinoleyl, behenyl, and erucyl alcohols, as well as 2-octyl dodecanol.

Specific examples of waxes suitable for use as emollients include lanolin and derivatives thereof including lanolin oil, lanolin wax, lanolin alcohols, lanolin fatty acids, isopropyl lanolate, ethoxylated lanolin, ethoxylated lanolin alcohols, ethoxolated cholesterol, propoxylated lanolin alcohols, acetylated lanolin, acetylated lanolin alcohols, lanolin alcohols linoleate, lanolin alcohols recinoleate, acetate of lanolin alcohols recinoleate, acetate of lanolin alcohols recinoleate, acetate of ethoxylated alcohols esters, hydrogenolysates of lanolin, hydrogenated lanolin, ethoxylated hydrogenated lanolin, ethoxylated sorbitol lanolin, and liquid and semisolid lanolin. Also usable as waxes include hydrocarbon waxes, ester waxes, and amide waxes. Useful waxes include wax esters such as beeswax, spermaceti, myristyl myristate and stearyl stearate; beeswax derivatives, e.g., polyoxyethylene sorbitol beeswax; and vegetable waxes including carnauba and candelilla waxes.

Polyhydric alcohols and polyether derivatives may be used as solvents and/or surfactants in the compositions. Suitable polyhydric alcohols and polyethers include propylene glycol, dipropylene glycol, polypropylene glycols 2000 and 4000, poly(oxyethylene-co-oxypropylene) glycols, glycerol, sorbitol, ethoxylated sorbitol, hydroxypropylsorbitol, polyethylene glycols 200-6000, methoxy polyethylene glycols 350, 550, 750, 2000 and 5000, poly[ethylene oxide] homopolymers (100,000-5,000,000), polyalkylene glycols and derivatives, hexylene glycol, 2-methyl-2,4-pentanediol, 1,3-butylene glycol, 1,2,6-hexanetriol, 2-ethyl-1,3-hexanediol, vicinal glycols having 15 to 18 carbon atoms, and polyoxypropylene derivatives of trimethylolpropane.

Polyhydric alcohol esters may be used as emulsifiers or emollients. Suitable polyhydric alcohol esters include ethylene glycol mono- and di-fatty acid esters, diethylene glycol mono- and di-fatty acid esters, polyethylene glycol (200-6000) mono- and di-fatty acid esters, propylene glycol mono- and di-fatty esters, polypropylene glycol 2000 monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters.

Suitable emulsifiers for use in compositions include anionic, cationic, nonionic, and zwitterionic surfactants. Preferred ionic emulsifiers include phospholipids, such as lecithin and derivatives.

Sterols including, for example, cholesterol and cholesterol fatty acid esters; amides such as fatty acid amides, ethoxylated fatty acid amides, and fatty acid alkanolamides may also be used as emollients and/or penetration enhancers.

A pharmaceutically acceptable preservative can be employed to increase the shelf life of the composition. Other suitable preservatives and/or antioxidants for use in compositions include benzalkonium chloride, benzyl alcohol, phenol, urea, parabens, butylated hydroxytoluene (BHT), butylated hydroxyanisole (BHA), tocopherol, thimerosal, chlorobutanol, or the like, and mixtures thereof, can be employed. If a preservative, such as an antioxidant, is employed, the concentration is typically from about 0.02% to about 2% based on the total weight of the composition, although larger or smaller amounts can be desirable depending upon the agent selected. Reducing agents, as described herein, can be advantageously used to maintain good shelf life of the composition. It is generally observed that the anhydrous compositions of the embodiments exhibit satisfactory stability, such that a preservative can be omitted from the composition.

Suitable chelating agents for use in compositions include ethylene diamine tetraacetic acid, alkali metal salts thereof alkaline earth metal salts thereof, ammonium salts thereof, and tetraalkyl ammonium salts thereof. In some embodiments, the chelating agent is disodium ethylenediaminetetraacetic acid (EDTA). In some embodiments, the disodium EDTA is provided at least or about 0.001 wt. %, 0.005 wt. %, 0.01 wt. %, 0.02 wt. %, 0.05 wt. %, 0.10 wt. %, 0.20 wt. %, 0.25 wt. %, 0.50 wt. %, 0.75 wt. %, 1.0 wt. %, 1.5 wt. %, 2.0 wt. %, 2.5 wt. %, 3.0 wt. %, 3.5 wt. %, 4.0 wt. %, or more than 4 wt. %. In some embodiments, the disodium EDTA is provided in a range of about 0.25 wt. % to about 10 wt. %, about 0.1 wt. % to about 2.5 wt. %, about 0.5 wt. % to about 8 wt. %, about 0.75 wt. % to about 6 wt. %, or about 1 wt. % to about 4 wt. %. In some embodiments, the disodium EDTA is provided in a range of about 0.001 wt. % to about 6 wt. %, about 0.002 wt. % to about 4 wt. %, about 0.01 wt. % to about 3 wt. %, or about 0.02 wt. % to about 2 wt. %.

The carrier preferably has a pH of between about 4.0 and 10.0, more preferably between about 4.8 and about 7.8, more preferably between about 5.0 to about 6.5. The pH may be controlled using buffer solutions or other pH modifying agents. Suitable pH modifying agents include phosphoric acid and/or phosphate salts, citric acid and/or citrate salts, hydroxide salts (i.e., calcium hydroxide, sodium hydroxide, potassium hydroxide) and amines, such as triethanolamine. Suitable buffer solutions include a buffer comprising a solution of monopotassium phosphate and dipotassium phosphate, maintaining a pH of between 5.8 and 8; and a buffer comprising a solution of monosodium phosphate and disodium phosphate, maintaining a pH of between 6 and 7.5. Other buffers include citric acid/sodium citrate, and dibasic sodium phosphate/citric acid. The peptide compositions of the embodiments are preferably isotonic with the blood or other body fluid of the recipient. The isotonicity of the compositions can be attained using sodium tartrate, propylene glycol or other inorganic or organic solutes. Sodium chloride is particularly preferred. Buffering agents can be employed, such as acetic acid and salts, citric acid and salts, boric acid and salts, and phosphoric acid and salts. It can be desirable to include a reducing agent in the composition, such as vitamin C, vitamin E, or other reducing agents as are known in the pharmaceutical arts.

Surfactants can also be employed as excipients, for example, anionic detergents such as sodium lauryl sulfate, dioctyl sodium sulfosuccinate and dioctyl sodium sulfonate, cationic such as benzalkonium chloride or benzethonium chloride, or nonionic detergents such as polyoxyethylene hydrogenated castor oil, glycerol monostearate, polysorbates, sucrose fatty acid ester, methyl cellulose, or carboxymethyl cellulose.

In some embodiments, it can be advantageous to include additional agents having pharmacological activity. Anti-infective agents include, but are not limited to, anthelmintic (mebendazole), antibiotics including aminoglycosides (gentamicin, neomycin, tobramycin), antifungal antibiotics (amphotericin b, fluconazole, griseofulvin, itraconazole, ketoconazole, nystatin, micatin, tolnaftate), cephalosporins (cefaclor, cefazolin, cefotaxime, ceftazidime, ceftriaxone, cefuroxime, cephalexin), beta-lactam antibiotics (cefotetan, meropenem), chloramphenicol, macrolides (azithromycin, clarithromycin, erythromycin), penicillins (penicillin G sodium salt, amoxicillin, ampicillin, dicloxacillin, nafcillin, piperacillin, ticarcillin), tetracyclines (doxycycline, minocycline, tetracycline), bacitracin, clindamycin, colistimethate sodium, polymyxin b sulfate, vancomycin, antivirals including acyclovir, amantadine, didanosine, efavirenz, foscarnet, ganciclovir, indinavir, lamivudine, nelfinavir, ritonavir, saquinavir, stavudine, valacyclovir, valganciclovir, zidovudine, quinolones (ciprofloxacin, levofloxacin), sulfonamides (sulfadiazine, sulfisoxazole), sulfones (dapsone), furazolidone, metronidazole, pentamidine, sulfanilamidum crystallinum, gatifloxacin, and sulfamethoxazole/trimethoprim. Anesthetics can include, but are not limited to, ethanol, bupivacaine, chloroprocaine, levobupivacaine, lidocaine, mepivacaine, procaine, ropivacaine, tetracaine, desflurane, isoflurane, ketamine, propofol, sevoflurane, codeine, fentanyl, hydromorphone, marcaine, meperidine, methadone, morphine, oxycodone, remifentanil, sufentanil, butorphanol, nalbuphine, tramadol, benzocaine, dibucaine, ethyl chloride, xylocaine, and phenazopyridine. Anti-inflammatory agents include but are not limited to, nonsteroidal anti-inflammatory drugs (NSAIDs) such as aspirin, celecoxib, choline magnesium trisalicylate, diclofenac potassium, diclofenac sodium, diflunisal, etodolac, fenoprofen, flurbiprofen, ibuprofen, indomethacin, ketoprofen, ketorolac, melenamic acid, nabumetone, naproxen, naproxen sodium, oxaprozin, piroxicam, rofecoxib, salsalate, sulindac, and tolmetin; and corticosteroids such as cortisone, hydrocortisone, methylprednisolone, prednisone, prednisolone, betamethesone, beclomethasone dipropionate, budesonide, dexamethasone sodium phosphate, flunisolide, fluticasone propionate, triamcinolone acetonide, betamethasone, fluocinonide, betamethasone dipropionate, betamethasone valerate, desonide, desoximetasone, fluocinolone, triamcinolone, clobetasol propionate, and dexamethasone.

In some embodiments, the addition of emollients, emulsion stabilizers, moisturizers, excipients, and other compounds may be modified to enhance the sensory properties of the topical compositions, including but not limited to: skin feel (such as silkiness, lightness, creaminess), absorbency (required time at which product loses wet feel and is no longer perceived on skin), consistency, firmness, spreadability (e.g. viscosity, flow onset, shear rates), stickiness, integrity of shape, glossiness, hydrophilicity or hydrophobicity, and others. Preferably, compositions will have high spreadability and low viscosity properties. Compositions with such properties have been demonstrated to have an enhanced “silky” or “light” skin feel rating (see e.g. Bekker, M. Webber, G., Louw, N. Relating rheological measurements to primary and secondary skin feeling when mineral-based and Fischer-Tropsch wax-based cosmetic emulsions and jellies are applied to the skin, International Journal of Cosmetic Science 2013, 35(4), pp. 354-61).

In some embodiments, compositions comprise polyacrylate-13, polyisobutene, polysorbate 20, or combinations thereof. In some embodiments, polyacrylate-13 is provided at least or about 0.05 wt. %, 0.10 wt. %, 0.25 wt. %, 0.50 wt. %, 0.75 wt. %, 1.0 wt. %, 1.5 wt. %, 2.0 wt. %, 2.5 wt. % 3.0 wt. %, 3.5 wt. %, 4.0 wt. %, 4.5 wt. %, 5.0 wt. %, 5.5 wt. %, 6.0 wt. %, 6.5 wt. % 7.0 wt. % 8 wt. %, 9 wt. %, 10 wt. %, or more than 10 wt. %. In some embodiments, polyacrylate-13 is provided in a range of about 0.25 wt. % to about 10 wt. %, about 0.5 wt. % to about 8 wt. %, about 0.75 wt. % to about 6 wt. %, or about 1 wt. % to about 4 wt. %. In some embodiments, polyisobutene is provided at least or about 0.05 wt. %, 0.10 wt. %, 0.25 wt. %, 0.50 wt. %, 0.75 wt. %, 1.0 wt. %, 1.5 wt. %, 2.0 wt. %, 2.5 wt. %, 3.0 wt. %, 3.5 wt. %, 4.0 wt. %, 4.5 wt. %, 5.0 wt. %, 5.5 wt. %, 6.0 wt. %, 6.5 wt. %, 7.0 wt. %, 8 wt. %, 9 wt. %, 10 wt. %, or more than 10 wt. %. In some embodiments, polyisobutene is provided in a range of about 0.25 wt. % to about 10 wt. %, about 0.5 wt. % to about 8 wt. %, about 0.75 wt. % to about 6 wt. %, or about 1 wt. % to about 4 wt. %. In some embodiments, polyacrylate-13 is provided in a range of about 0.25 wt. % to about 10 wt. %, about 0.5 wt. % to about 8 wt. %, about 0.75 wt. % to about 6 wt. %, or about 1 wt. % to about 4 wt. %. In some embodiments, polysorbate 20 is provided at least or about 0.05 wt. %, 0.10 wt. %, 0.25 wt. %, 0.50 wt. %, 0.75 wt. %, 1.0 wt. %, 1.5 wt. %, 2.0 wt. %, 2.5 wt. %, 3.0 wt. %, 3.5 wt. %, 4.0 wt. %, 4.5 wt. %, 5.0 wt. %, 5.5 wt. %, 6.0 wt. %, 6.5 wt. %, 7.0 wt. %, 8 wt. %, 9 wt. %, 10 wt. %, or more than 10 wt. %. In some embodiments, polysorbate 20 is provided in a range of about 0.25 wt. % to about 10 wt. %, about 0.5 wt. % to about 8 wt. %, about 0.75 wt. % to about 6 wt. %, or about 1 wt. % to about 4 wt. %. In some embodiments, polyacrylate-13, polyisobutene, and polysorbate 20 are provided at least or about 0.05 wt. %, 0.10 wt. %, 0.25 wt. %, 0.50 wt. %, 0.75 wt. %, 1.0 wt. %, 1.5 wt. %, 2.0 wt. %, 2.5 wt. %, 3.0 wt. %, 3.5 wt. %, 4.0 wt. %, 4.5 wt. %, 5.0 wt. %, 5.5 wt. %, 6.0 wt. %, 6.5 wt. %, 7.0 wt. %, 8 wt. %, 9 wt. %, 10 wt. %, or more than 10 wt. %. In some embodiments, polyacrylate-13, polyisobutene, and polysorbate 20 are provided in a range of about 0.25 wt. % to about 10 wt. %, about 0.1 wt. % to about 4 wt. %, about 0.5 wt. % to about 8 wt. %, about 0.75 wt. % to about 6 wt. %, or about 1 wt. % to about 4 wt. %.

The topical composition may contain micelles, or an aggregate of surfactant molecules dispersed in an aqueous solution. Micelles may be prepared by dispersing an oil solvent in an aqueous solution comprising a surfactant, where the surfactant concentration exceeds the critical micelle concentration. The resulting composition contains micelles, i.e., spherical oil droplets

Penetration Enhancers

Fatty acids and alcohols can be employed to enhance penetration of the peptides, and to provide a silky feel to compositions, e.g., methanoic acid, ethanoic acid, propanoic acid, butanoic acid, isobutyric acid, pentanoic acid, hexanoic acid, heptanoic acid, octanoic acid, nonanoic acid, decanoic acid, myristoleic acid, isovaleric acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, docosahexaenoic acid, caprylic acid, capric acid, lauric acid, palmitic acid, stearic acid, arachidic acid, behenic acid, lignoceric acid, cerotic acid, medium chain fatty acids, e.g., C₆₋₁₂ fatty acids, or the like. Typical amounts when employed in compositions are from 1 wt. % to 4 wt. %.

### Methods of Use

Described herein are topical compositions and methods of use for body contouring or surgical procedure. In some embodiments, the topical compositions are used for pre-conditioning. In some embodiments, the topical compositions are used after a body contouring or surgical procedure. In some embodiments, the topical compositions are used both for preparation for a body contouring or surgical procedure and after a body contouring or surgical procedure.

Compositions as described herein, in some embodiments, comprise lactoferrin, tripeptide-1, hexapeptide-12, hexapeptide-11, phosphatidylserine, tetrapeptide-2, Peucedanum graveolens extract, hydroxymethoxyphenyl decanone, Dunaliella salina extract, Tremella extract, betaine, Ledum palustre extract, xylitylglucoside, anhydroxylitol, xylitol, oleuropein, Centella asiatica extract, naringenin, Arnica montana flower extract, tetrandrine, or combinations thereof. In some embodiments, the compositions described herein comprise in some embodiments, comprise lactoferrin, tripeptide-1, hexapeptide-12, hexapeptide-11, phosphatidylserine, tetrapeptide-2, Peucedanum graveolens extract, hydroxymethoxyphenyl decanone, Dunaliella salina extract, Tremella extract, betaine, Ledum palustre extract, xylitylglucoside, anhydroxylitol, xylitol, oleuropein, Centella asiatica extract, naringenin, Arnica montana flower extract, and tetrandrine.

In some embodiments, compositions as described herein are used to improve or prevent effects associated with a body contouring or surgical procedure. In some embodiments, the topical compositions are used to improve patient report recovery outcome measures (PROM) associated with a body contouring or surgical procedure. In some embodiments, compositions as described herein are used to improve or prevent effects associated with a body contouring or surgical procedure including, but not limited to, bruising, swelling, fat dissolution, antimicrobial effects, skin tightening, and scarring. In some embodiments, compositions as described herein promote skin regeneration. In some embodiments, compositions as described herein modulate extracellular remodeling. In some embodiments, compositions as described herein promote wound healing. In some embodiments, compositions as described herein are useful in improving bruising. Compositions and methods as described herein, in some embodiments, improve bruising by removing by-products of red blood cell extravasation more efficiently. In some embodiments, compositions as described herein reduce swelling. In some embodiments, compositions as described herein reduce skin discoloration, ecchymosis, edema, induration, subcutaneous fibrous banding, pain, and combinations thereof. In some embodiments, compositions as described herein reduce pain. In some embodiments, compositions as described herein promote anti-microbial effects. In some embodiments, compositions as described herein improve function of macrophages. In some embodiments, compositions as described herein stimulate elastin production. In some embodiments, compositions as described herein aid in elimination of lipid droplets. In some embodiments, compositions as described herein stimulate autophagy. In some embodiments, compositions as described herein stimulate autophagy and macrophage production to accelerate digestion of fat fragments. In some embodiments, compositions as described herein are used to improve healing. In some embodiments, compositions as described herein improve healing by modulating adipocytolysis.

In some embodiments, compositions described herein improve or prevent scarring by modulating various signaling pathways. In some embodiments, the compositions described herein modulate signaling pathways involved in mechanostimulation, scar support, hydration occlusion, inflammation, and collagen/extracellular remodeling.

In some embodiments, compositions described herein improve or prevent scarring by modulating gene expression. In some embodiments, compositions modulate expression of TGF-β1, TGF-β3, IL-10, IL-6, SMAD-2, SMAD-3, SMAD-4, SMAD-7, COX-2, IL-β, TNF-α, tissue inhibitors of metalloproteinase (TIMP), MMP-1, VEGF, or combinations thereof. In some embodiments, compositions as described herein decrease expression of TGF-β1, TGF-β3, IL-10, IL-6, SMAD-2, SMAD-3, SMAD-4, SMAD-7, COX-2, IL-β, TNF-α, tissue inhibitors of metalloproteinase (TIMP), MMP-1, VEGF, or combinations thereof by at least or about 10%, 15%, 20%, 25%, 30%, 35%, 40%, 45%, 50%, 55%, 60%, 65%, 70%, 75%, 80%, 85%, 90%, 95%, or more than 95%. In some embodiments, compositions as described herein decrease expression of TGF-β1, TGF-β3, IL-10, IL-6, SMAD-2, SMAD-3, SMAD-4, SMAD-7, COX-2, IL-β, TNF-α, tissue inhibitors of metalloproteinase (TIMP), MMP-1, VEGF, or combinations thereof by at least or about 0.5×, 1.0×, 1.5×, 2.0×, 2.5×, 3.0×, 3.5×, 4.0×, 5.0×, 6.0×, 7.0×, 8.0×, 9.0×, 10×, or more than 10×. In some embodiments, compositions modulate expression of TGF-β. In some embodiments, compositions as described herein decrease expression of TGF-β. In some embodiments, compositions as described herein decrease expression of TGF-β by at least or about 10%, 15%, 20%, 25%, 30%, 35%, 40%, 45%, 50%, 55%, 60%, 65%, 70%, 75%, 80%, 85%, 90%, 95%, or more than 95%. In some embodiments, compositions as described herein decrease expression of TGF-β by at least or about 0.5×, 1.0×, 1.5×, 2.0×, 2.5×, 3.0×, 3.5×, 4.0×, 5.0×, 6.0×, 7.0×, 8.0×, 9.0×, 10×, or more than 10×. In some embodiments, TGF-β is TGF-β1. In some embodiments, TGF-β is TGF-β3. In some embodiments, compositions modulate expression of SMAD-7. In some embodiments, compositions as described herein increase expression of SMAD-7. In some embodiments, compositions as described herein increase expression of SMAD-7 by at least or about 10%, 15%, 20%, 25%, 30%, 35%, 40%, 45%, 50%, 55%, 60%, 65%, 70%, 75%, 80%, 85%, 90%, 95%, or more than 95%. In some embodiments, compositions as described herein increase expression of SMAD-7 by at least or about 0.5×, 1.0×, 1.5×, 2.0×, 2.5×, 3.0×, 3.5×, 4.0×, 5.0×, 6.0×, 7.0×, 8.0×, 9.0×, 10×, or more than 10×. In some embodiments, compositions as described herein decrease expression of TGF-β1 and increase expression of TGF-β3, SMAD-7, or both. In some embodiments, compositions as described herein decrease expression of TGF-β1 by at least or about 10%, 15%, 20%, 25%, 30%, 35%, 40%, 45%, 50%, 55%, 60%, 65%, 70%, 75%, 80%, 85%, 90%, 95%, or more than 95% and increase expression of TGF-β3, SMAD-7, or both by at least or about 10%, 15%, 20%, 25%, 30%, 35%, 40%, 45%, 50%, 55%, 60%, 65%, 70%, 75%, 80%, 85%, 90%, 95%, or more than 95%. In some embodiments, compositions as described herein decrease expression of TGF-β1 by at least or about 0.5×, 1.0×, 1.5×, 2.0×, 2.5×, 3.0×, 3.5×, 4.0×, 5.0×, 6.0×, 7.0×, 8.0×, 9.0×, 10×, or more than 10× and increase expression of TGF-β3, SMAD-7, or both by at least or about 0.5×, 1.0×, 1.5×, 2.0×, 2.5×, 3.0×, 3.5×, 4.0×, 5.0×, 6.0×, 7.0×, 8.0×, 9.0×, 10×, or more than 10×.

In some embodiments, compositions described herein improve resolution of inflammation and/or induration. In some embodiments, compositions described herein improve resolution of inflammation and/or induration by modulating gene expression. In some embodiments, composition described herein modulate gene expression of TNF-α, IL-1, IL-6, or combinations thereof. In some embodiments, composition described herein modulate gene expression of TNF-α. In some embodiments, compositions as described herein increase expression of TNF-α. In some embodiments, compositions as described herein decrease expression of TNF-α. In some embodiments, compositions as described herein increase or decrease expression of TNF-α by at least or about 10%, 15%, 20%, 25%, 30%, 35%, 40%, 45%, 50%, 55%, 60%, 65%, 70%, 75%, 80%, 85%, 90%, 95%, or more than 95%. In some embodiments, composition described herein modulate gene expression of IL-1. In some embodiments, compositions as described herein increase expression of IL-1. In some embodiments, compositions as described herein decrease expression of IL-1. In some embodiments, compositions as described herein increase or decrease expression of IL-1 by at least or about 10%, 15%, 20%, 25%, 30%, 35%, 40%, 45%, 50%, 55%, 60%, 65%, 70%, 75%, 80%, 85%, 90%, 95%, or more than 95%. In some embodiments, composition described herein modulate gene expression of IL-6. In some embodiments, composition described herein modulate gene expression of IL-1. In some embodiments, compositions as described herein increase expression of IL-6. In some embodiments, compositions as described herein decrease expression of IL-6. In some embodiments, compositions as described herein increase or decrease expression of IL-6 by at least or about 10%, 15%, 20%, 25%, 30%, 35%, 40%, 45%, 50%, 55%, 60%, 65%, 70%, 75%, 80%, 85%, 90%, 95%, or more than 95%.

Compositions as described herein may be used with various body contouring or surgical procedures. In some embodiments, the body contouring or surgical procedure is an invasive procedure. In some instances, the invasive procedure comprises use of an invasive laser or surgery. In some embodiments, the body contouring or surgical procedure is a non-invasive procedure. In some instances, the non-invasive procedure comprises non-surgical skin tightening, non-surgical fat reduction, or use of a non-invasive laser.

In some embodiments, the body contouring or surgical procedure comprises abdominoplasty, liposuction, bariatric surgery, breast reduction, breast augmentation, breast lift, breast reconstruction, thigh lift, fat reduction, forehead lift, cheek enhancement, otoplasty, rhytidectomy, lower rhytidectomy, cheek reduction, mentoplasty, blepharoplasty, facial implant, nose surgery, skin excision, skin biopsy, invasive cellulite treatment, or combinations thereof. In some embodiments, the abdominoplasty is performed without liposuction. In some embodiments, the abdominoplasty is performed with liposuction. In some embodiments, the liposuction is of the body. In some embodiments, the liposuction is of the neck. In some embodiments, the fat reduction is in the abdomen. In some instances, the fat reduction is in the arm. In some instances, the fat reduction is in a submental region, abdomen, face, flank, back, chest, arm, leg, buttock, or combination thereof. In some embodiments, the body contouring or surgical procedure comprises a fat grafting procedure. In some embodiments, the the body contouring or surgical procedure comprises a reconstructive procedure. In some embodiments, the reconstructive procedure comprises tissue-based reconstruction. In some embodiments, the reconstructive procedure comprises a tissue flap procedure (autologous tissue reconstruction). In some embodiments, the body contouring or surgical procedure comprises low level laser therapy, infrared light, high frequency focused ultrasound, pulsed focus ultrasound, radiofrequency, radiofrequency induced electroporation, injectable lipolytic agents, cryolipolysis, or combinations thereof. In some instances, the body contouring or surgical procedure comprises an energy source. In some instances, the energy source is electromagnetic energy. In some instances, the procedure is high intensity focused electro-magnetic technology (HIFEM).

In some embodiments, the body contouring or surgical procedure comprises an injection of a filler. In some embodiments, the filler is a soft tissue filler product. For example, the soft tissue filler is an injectable dermal or subdermal filler. In some embodiments, the filler is a breast augmentation or reconstruction filler, a lip filler, or filler suitable for other soft tissue restoration or augmentation. In some embodiments, the filler is dermal filler. In some instances, the dermal filler is administered through injection into or beneath the skin of a subject.

In some embodiments, the body contouring or surgical procedure comprises injection of an injectable such as Botox®, Dysport®, or Xeomin®.

In some embodiments, the body contouring or surgical procedure comprises injection of an agent or composition that reduces cellulite. In some embodiments, the agent or composition comprises an enzyme. In some embodiments, the agent or composition comprises collagenase. In some embodiments, the injection of an agent or composition that reduces cellulite comprises injection of Qwo™.

In some embodiments, the body contouring or surgical procedure is microneedling. In some instances, the body contouring or surgical procedure is radiofrequency microneedling. In some embodiments, the body contouring or surgical procedure comprises cellulite reduction or enzymatic (collagenase) or mechanical disruption of fascial bands.

In some embodiments, the body contouring or surgical procedure comprises a procedure to reduce visibility of veins. In some embodiments, the visible vein is a varicose vein. Exemplary procedures to reduce visibility of veins include, but are not limited to, post vein sclerotherapy, laser treatment, endovenous laser therapy (EVLT), radiofrequency ablation (RFA), catheter-assisted procedures using radiofrequency or laser energy, high ligation and vein stripping, ambulatory phlebectomy, and endoscopic vein surgery.

In some instances, the topical compositions described herein are administered once per day, twice per day, three times per day or more. In some instances, the topical compositions described herein are administered twice per day. The topical compositions described herein, in some embodiments, are administered daily, every day, every alternate day, five days a week, once a week, every other week, two weeks per month, three weeks per month, once a month, twice a month, three times per month, or more. In some embodiments, the topical compositions described herein are administered twice daily, e.g., morning and evening. In some embodiments, the topical compositions described herein are administered for at least 1 day, 2 days, 3 days, 4 days, 5 days, 6 days, 1 week, 2 weeks, 3 weeks, 1 month, 2 months, 3 months, 4 months, 5 months, 6 months, 7 months, 8 months, 9 months, 10 months, 11 months, 12 months, 18 months, 2 years, 3 years, 4 years, 5 years, 10 years, or more. In some embodiments, the topical compositions described herein are administered twice daily for at least or about 1 week, 2 weeks, 3 weeks, 1 month, 2 months, 3 months, 4 months, 5 months, 6 months, or more. In some embodiments, the topical compositions described herein are administered once daily, twice daily, three times daily, four times daily, or more than four times daily for at least or about 1 week, 2 weeks, 3 weeks, 1 month, 2 months, 3 months, 4 months, 5 months, 6 months, or more.

Various symptoms or effects of any body contouring or surgical procedure can be improved with the use of the methods described herein. In some embodiments, the formulations described herein are used in conjunction with a fat reduction procedure. In some embodiments, the fat reduction procedure is liposuction. In some instances, the reduction is in the abdomen. In some instances, the reduction is in the arm. In some instances, the reduction is in a submental region, abdomen, face, flank, back, chest, arm, leg, buttock, or combination thereof.

In some instances, the formulations described herein are administered prior to a body contouring or surgical procedure. In additional instances, the formulations described herein are administered as a pre-conditioning treatment. In some cases, the topical compositions described herein are administered up to 1 hour, up to 2 hours, up to 3 hours, up to 5 hours, up to 6 hours, up to 7 hours, up to 8 hours, up to 12 hours, up to 16 hours, up to 20 hours, or up to 24 hours prior to a body contouring or surgical procedure. In some instances, the topical formulation described herein are administered for at least 1 day, 2 days, 3 days, 4 days, 5 days, 6 days, 1 week, 2 weeks, 3 weeks, 1 month, 2 months, 3 months, 4 months, 5 months, 6 months, or more prior to a body contouring or surgical procedure. In some instances, the topical compositions described herein are administered for at least 2-8 weeks, 2-6 weeks, 2-4 weeks, or 2-3 weeks as a pre-conditioning treatment. In some cases, the formulations described herein are administered at least 1 day, 2 days, 3 days, 4 days, 5 days, 6 days, 1 week, 2 weeks, 3 weeks, 1 month, 2 months, 3 months, 4 months, 5 months, 6 months, or more prior to a body contouring or surgical procedure. Sometimes the formulations described herein are administered singly, or over a time course, such as daily, multiple times weekly, weekly, biweekly, monthly or less frequently prior to a body contouring or surgical procedure. In some instances, the formulations described herein are administered singly, or over a time course, such as daily, multiple times weekly, weekly, biweekly, monthly or more frequently prior to a body contouring or surgical procedure. In some instances, the topical compositions described herein are administered once per day, twice per day, three times per day or more prior to a body contouring or surgical procedure. In some instances, the topical compositions described herein are administered twice daily administration, e.g., morning and evening, prior to a body contouring or surgical procedure.

In some instances, the formulations described herein are administered follow a body contouring or surgical procedure. In some instances, the formulations described herein are administered up to 1 hour, up to 2 hours, up to 3 hours, up to 5 hours, up to 6 hours, up to 7 hours, up to 8 hours, up to 12 hours, up to 16 hours, up to 20 hours, or up to 24 hours after a body contouring or surgical procedure. In some cases, the formulations described herein are administered at least or up to 1 day, 2 days, 3 days, 4 days, 5 days, 6 days, 1 week, 2 weeks, 3 weeks, 1 month, 2 months, 3 months, 4 months, 5 months, 6 months, or more after a body contouring or surgical procedure. In some embodiments, the topical compositions described herein are administered for at least 2-8 weeks, 2-6 weeks, 2-4 weeks, or 2-3 weeks after a body contouring or surgical procedure. Sometimes the formulations described herein are administered singly, or over a time course, such as daily, multiple times weekly, weekly, biweekly, monthly or less frequently after a body contouring or surgical procedure. In some instances, the formulations described herein are administered singly, or over a time course, such as daily, multiple times weekly, weekly, biweekly, monthly or more frequently after a body contouring or surgical procedure. In some instances, the topical compositions described herein are administered once per day, twice per day, three times per day or more after a body contouring or surgical procedure. In some instances, the topical compositions described herein are administered twice daily administration, e.g., morning and evening, after a body contouring or surgical procedure.

In some instances, the formulations described herein are administered prior to a body contouring or surgical procedure and after a body contouring or surgical procedure. In additional instances, the formulations described herein are administered as a pre-conditioning treatment. In some instances, the topical formulation described herein are administered for at least 1 day, 2 days, 3 days, 4 days, 5 days, 6 days, 1 week, 2 weeks, 3 weeks, 1 month, 2 months, 3 months, 4 months, 5 months, 6 months, or more as a pre-conditioning treatment. In some instances, the topical compositions described herein are administered for at least 2-8 weeks, 2-6 weeks, 2-4 weeks, or 2-3 weeks as a pre-conditioning treatment. In some cases, the topical compositions described herein are administered up to 1 hour, up to 2 hours, up to 3 hours, up to 5 hours, up to 6 hours, up to 7 hours, up to 8 hours, up to 12 hours, up to 16 hours, up to 20 hours, or up to 24 hours prior to a body contouring or surgical procedure and administered up to 1 hour, up to 2 hours, up to 3 hours, up to 5 hours, up to 6 hours, up to 7 hours, up to 8 hours, up to 12 hours, up to 16 hours, up to 20 hours, or up to 24 hours after a body contouring or surgical procedure. In some cases, the formulations described herein are administered at least 1 day, 2 days, 3 days, 4 days, 5 days, 6 days, 1 week, 2 weeks, 3 weeks, 1 month, 2 months, 3 months, 4 months, 5 months, 6 months, or more prior to a body contouring or surgical procedure and administered at least or up to 1 day, 2 days, 3 days, 4 days, 5 days, 6 days, 1 week, 2 weeks, 3 weeks, 1 month, 2 months, 3 months, 4 months, 5 months, 6 months, or more after a body contouring or surgical procedure. In some embodiments, the topical compositions described herein are administered for at least 2-8 weeks, 2-6 weeks, 2-4 weeks, or 2-3 weeks prior to a body contouring or surgical procedure and are administered for at least 2-8 weeks, 2-6 weeks, 2-4 weeks, or 2-3 weeks after a body contouring or surgical procedure.

### Stability Testing

Stability testing of the compositions can be conducted as follows.

High temperature testing is now commonly used as a predictor of long-term stability. High temperature testing can be conducted at 37° C. (98° F.) and 45° C. (113° F.). If a product is stored at 45° C. for three months (and exhibits acceptable stability) then it should be stable at room temperature for two years. A good control temperature is 4° C. (39° F.) where most products will exhibit excellent stability. Sometime, the product is also be subjected to −10° C. (14° F.) for three months.

In some instances, stability of the product is assessed by passing three cycles of temperature testing from −10° C. (14° F.) to 25° C. (77° F.). In such cases, the product is placed at −10° C. for 24 hours and then placed at room temperature (25° C.) for 24 hours. This completes one cycle. An even more rigorous test is a −10° C. to 45° C. five-cycle test. This puts emulsions under a tremendous stress.

The dispersed phase (of an oil-in-water emulsion) has a tendency to separate and rise to the top of the emulsion forming a layer of oil droplets. This phenomenon is called creaming. Creaming is one of the first signs of impending emulsion instability. A test method to predict creaming is centrifugation. Heat the emulsion to 50° C. (122° F.) and centrifuge it for thirty minutes at 3000 rpm. Then inspect the resultant product for signs of creaming.

Both formulas and packaging can be sensitive to the UV radiation. The product is placed in glass and the actual package in a light box that has a broad-spectrum output. Another glass jar completely covered with aluminum foil serves as a control. Discoloration of the product may be observed.

For all the above-mentioned tests, the color, odor/fragrance, viscosity, pH value, and, if available, particle size uniformity and/or particle agglomeration under the microscope can be observed.

### Kits for Non-Invasive Use

Some embodiments of the methods and compositions provided herein include kits comprising peptides provided herein. In some embodiments, kits can be provided to an administering physician, other health care professional, a patient, or a caregiver. In some embodiments, a kit comprises a container which contains the peptide compositions in a suitable topical composition, and instructions for administering the peptide composition to a subject. The kit can optionally also contain one or more additional therapeutic or other agents. For example, a kit containing a peptide composition in topical form can be provided along with other skin care agents, such as, cleansers, occlusive moisturizers, penetrating moisturizers, sunscreens, sunblocks, and the like. The kit may contain the peptide composition in bulk form, or can contain separate doses of the peptide composition for serial or sequential administration. The kit can optionally contain one or more diagnostic tools, administration tools, and/or instructions for use. The kit can contain suitable delivery devices, such as, syringes, pump dispensers, single dose packets, and the like, along with instructions for administering the peptide compositions and any other therapeutic or beneficial agents. The kit can optionally contain instructions for storage, reconstitution (if applicable), and administration of any or all therapeutic or beneficial agents included. The kits can include a plurality of containers reflecting the number of administrations to be given to a subject, or the different products to be administered to the subject.

In some embodiments, the composition also works with the skin's own natural regenerating process and assists in improving the skin's appearance and skin tightness. The topical composition is suitable for all skin types and post-procedure skin. The topical compositions can be provided to the patient in bulk form, to permit a suitable amount of the peptides to be self-administered by the patient. For example, the patient can apply an amount of the composition sufficient to provide an even coating over the affected area or as otherwise instructed by the physician. In some embodiments it can desirable to incorporate additional therapeutic or active agents into the topical composition. Alternatively, adjunct therapies or agents can be administered separately. For example, a cleanser, a sunblock, a sunscreen, a penetrating moisturizer, and/or an occlusive moisturizer can be provided for administration before or after the topical composition of the embodiments.

The various examples of creams, ointments, lotions, solutions, gels, sprays and patches may incorporate the peptide compositions as described herein as the active ingredient, in combination with penetration enhancing agents and other active agents acting synergistically on the skin for the promotion of wound healing or wound closure or the treatment of chronic cutaneous wound.

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1: Exemplary Compositions

An exemplary composition is seen in Table 1.

TABLE 1
Active ingredients in exemplary composition

| | Concentration |
|---|---|
| Gene | (% by weight) |
| Lactoferrin | 0.01-0.25 |
| Tripeptide-1 | 0.6-15 |
| Hexapeptide-12 | 0.6-15 |
| Hexapeptide-11 | 0.002-0.05 |
| Phosphatidylserine | 0.02-0.5 |
| Tetrapeptide-2 | 0.2-5 |
| Peucedanum Graveolens Extract | 0.1-2.5 |
| Hydroxymethoxyphenyl Decanone | 0.1-2.5 |
| Dunaliella Salina Extract | 0.1-2.5 |
| Tremella Extract, Betaine | 0.1-2.5 |
| Ledum Palustre Extract | 0.1-2.5 |
| Aquaxyl (Xylitylglucoside, Anhydroxylitol, Xylitol) | 0.2-5 |
| Oleuropein | 0.01-0.25 |
| Centella asiatica extract | 0.2-5 |
| Naringenin | 0.4-10 |
| Arnica montana flower extract | 0.1-2.5 |
| Tetrandrine | 0.0002-0.005 |

### Example 2. Gene Expression Studies

Gene expression in fibroblasts were measured following 24 hours of exposure to tripeptide-1, hexapeptide-12, and tripeptide-1 and hexapeptide-12.

Briefly, primary human adult dermal cell lines were treated with tripeptide-1, hexapeptide-12, tripeptide-1 and hexapeptide-12, and DMSO (control). The dermal cells included primary fibroblasts, keratinocytes, and melanocytes (ZenBio). Primary cells were plated in 48 well plates at 5000 or 10000 cells per cm² as per ZenBio's instructions with cell specific media. Media volume included 500 microliter per well. Cells were cultured for 2 days in an incubator (37° C. with 5% CO²). After 48 hours, the cells were treated with tripeptide-1, hexapeptide-12, tripeptide-1 and hexapeptide-12, or DMSO. The cells were treated for 24 hours. Following 24 hours, the media was removed and the cells were washed 1× with PBS. 100 uL of RNA Lysis Buffer (Takara Bio Cat No. 635013, “10×RNA lysis buffer” diluted to 1×) was added to the well and mixed thoroughly by trituration, combined in RNAse microcentrifuge tubes, and immediately frozen at −30° C. RNA was extracted from the samples. For each sample, the library was constructed and sequences to 25M paired end 100 bp reads.

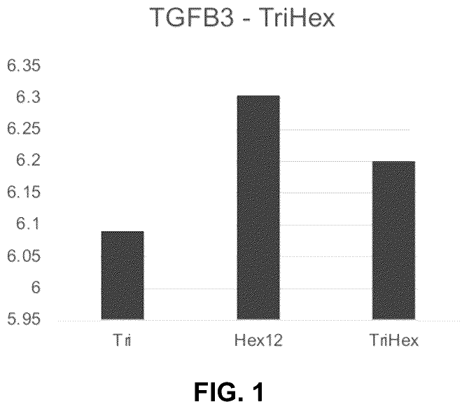

Data is seen in FIG. 1. FIG. 1 shows the gene expression for TGF-β3.

### Example 3: Pre-Conditioning Following Surgery

Pre-treatment was undertaken with different products (control moisturizing agent one side and TriHex technology other) for two weeks prior to surgery. Subjects were then treated for 10 weeks post-surgery and the resolution of skin changes were compared. The liposuction was carried out on medial thighs with a volume that was consistent at 250 mL per medial thigh with similar suction techniques by one surgeon.

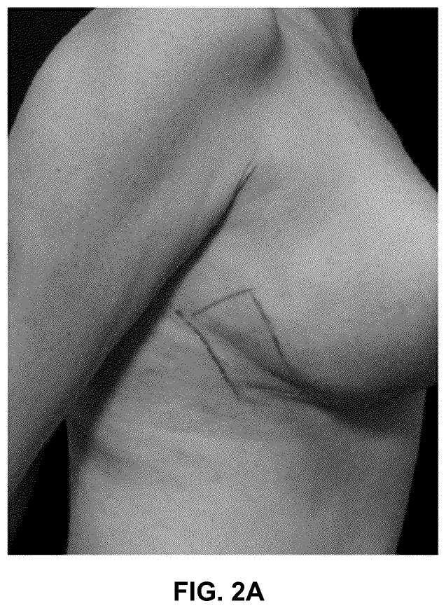

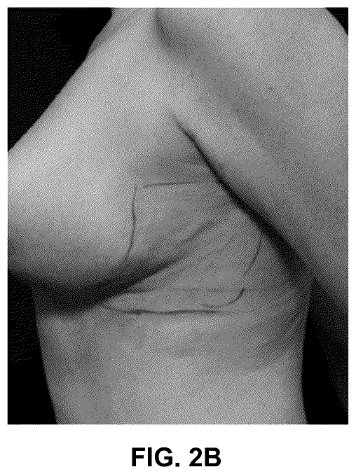

The data shows pre-treatment with a product comprising tripeptide-1 and hexapeptide-12 improved resolution of inflammation and induration (FIGS. 2A-2B).

This Example shows that a product incorporating tripeptide-1 and hexapeptide-12 similar to the composition in Table 1 can improve post-surgical healing.

### Example 4: Topical Compositions for Surgery

A multicenter patient experience trial is conducted. Five consecutive female patients at each of 4 centers are subject to a body contouring procedure. Patients are subject to either the surgical product having a composition as set forth in Table 1 or a bland moisturizer. Patient-reported scores of five skin parameters (skin discoloration, ecchymosis, edema, induration, subcutaneous fibrous banding) and pain scores using the Visual Analog Scale (VAS) are collected at eight different intervals for 12 weeks post-procedure.

Patients that receive the exemplary composition of Table 1 experience improved symptoms and recovery.

### Example 5: Bilateral Medial Thigh Liposuction

This example assesses the effect of the exemplary composition of Table 1 on a patient's medial thigh post-liposuction.

Patients are selected and randomized to apply the exemplary composition or bland moisturizer on the medial thigh following bilateral medial thigh liposuction. Patients are assessed using skin fibrometer measurements, ultrasounds, blinded investigator assessments, participant assessments, photography, and biopsies. Patients that receive the exemplary composition experience improved symptoms and recovery.

### Example 6: Multicenter Trial

A multicenter patient experience trial was conducted at 7 US sites and 1 Canadian site. Patients were subject to either the surgical product having a composition as set forth in Table 1 or a bland moisturizer. The patients underwent a surgical procedure prior to application of the composition or the moisturizer. The surgical procedures for body and neck included abdominoplasty, liposuction (PAL, mechanical, UAL), liposuction with J-plasma, and bilateral limited incision brachioplasty (axillary). The surgical procedures for the breast included breast augmentation, bilateral breast reduction, bilateral mastopexy, capsulectomy with implant exchange, and mastopexy with augmentation. Physician and Participant assessment of ecchymosis, skin discoloration, subcutaneous banding, induration, edema and pain were measured following 12 weeks post procedure. Photography (including 3D), ultrasound (bilateral pre and post-surgery), and biopsies (bilateral pre and post 2 weeks at surgery) were also taken. Usually, skin discoloration may be characterized by red/brown color on the skin or incision. Often, edema may be characterized by swelling. Typically, ecchymosis may be characterized by bruising. In some cases, the induration may be characterized by skin hardening.

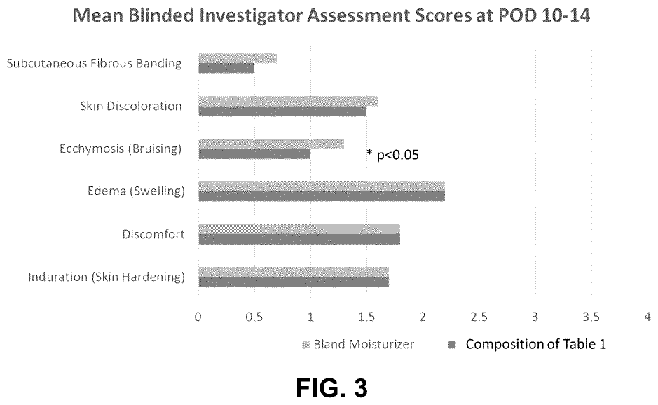

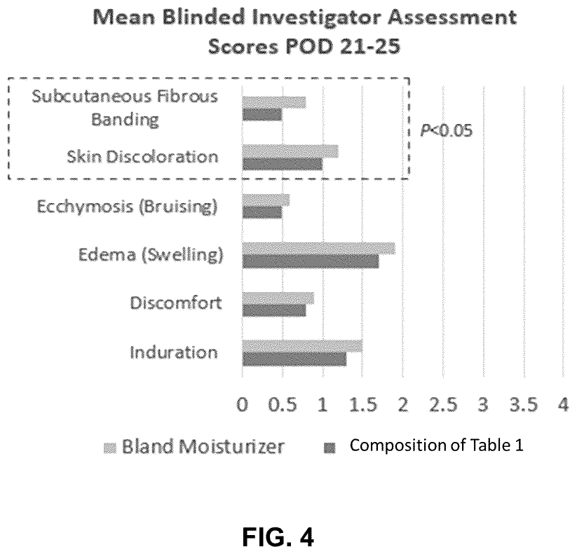

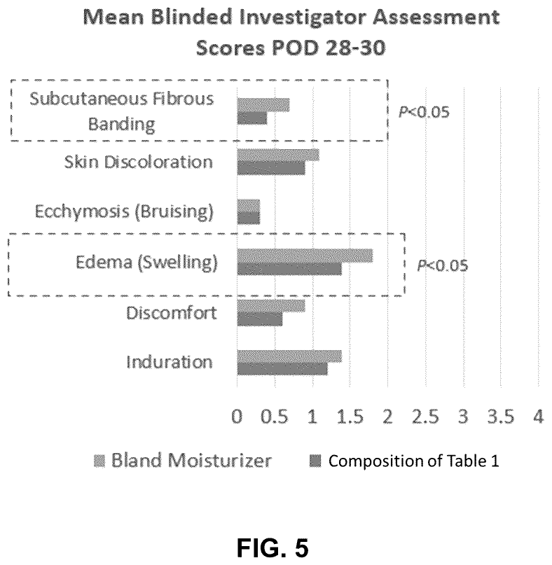

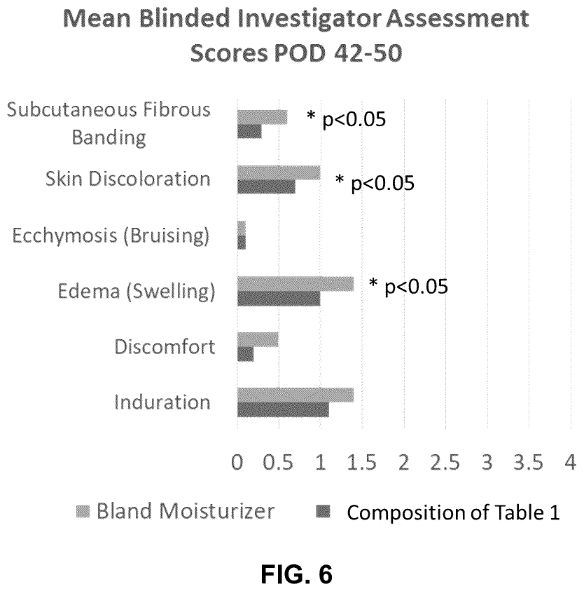

Data (n=35) of investigator assessments indicated there were no major differences first week. Use of a composition as set forth in Table 1 resulted in improvements in ecchymosis (postoperative day (POD) 10-14), skin discoloration (POD 21-25, POD 42-50), subcutaneous fibrous banding (POD 21-25, POD 28-30, POD 42-50), and edema (POD 28-30, POD 42-50) at various time points in the post-operative period. FIG. 3 illustrates that the use of the composition of Table 1 significantly improved ecchymosis at POD 10-14 as compared to the bland moisturizer as assessed by mean blinded investigator assessment scores, where p<0.05. The mean blinded investigator assessment scores were similar for subcutaneous fibrous banding, skin discoloration, edema, VAS, and induration at POD 10-14. FIG. 4 shows the mean blinded investigator assessment scores at POD 21-25. The use of the composition of Table 1 resulted in significantly improved subcutaneous fibrous banding and skin discoloration at POD 21-25 (p<0.05). FIG. 5 shows the mean blinded investigator assessment scores at POD 28-30. The use of the composition of Table 1 resulted in significantly improved subcutaneous fibrous banding and edema at POD 28-30 (p<0.05). FIG. 6 shows the mean blinded investigator assessment scores at POD 42-50. The use of the composition of Table 1 resulted in significantly improved subcutaneous fibrous banding, skin discoloration, and edema at POD 42-50 (p<0.05).

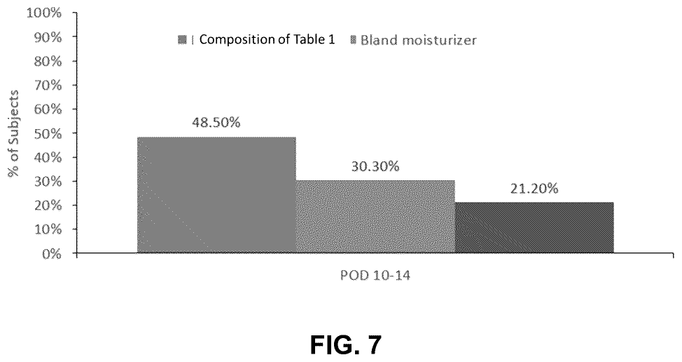

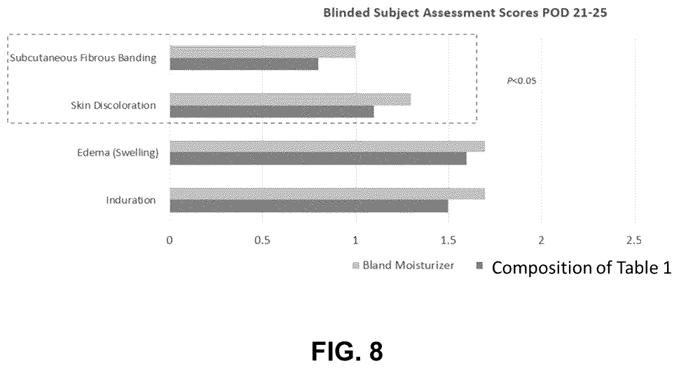

Data from participant questionnaires indicated that use of a composition as set forth in Table 1 resulted in less bruising and discoloration at POD 10-14 and POD 21-25, more soft and flexible at POD 28-30, and less swelling at POD 28-30. FIG. 7 shows that a statistically significant proportion of subjects reported less bruising and discoloration on the side treated with the composition of Table 1 (48.5% of subjects) as compared to the side treated with bland moisturizer (30.3% of subjects), with a p<0.0001 at all follow-up time points. 21.2% of the subjects reported no difference. This may indicate accelerated healing on the side treated with the composition. FIG. 8 shows that the mean blinded subject scores of the group treated with the composition of Table 1 demonstrated a significant improvement over bland moisturizer in subcutaneous fibrous banding (1 for bland moisturizer vs 0.8 for the composition) and skin discoloration (1.3 vs 1.1) at POD 21-25.

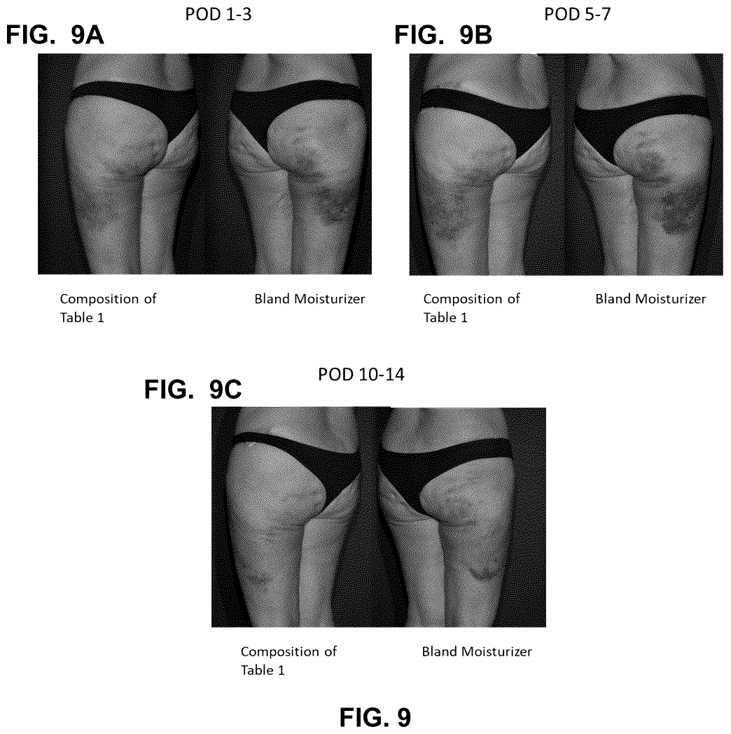

FIGS. 9A-C show images of the hips and thighs of a 62 years old subject after a lateral thigh liposuction who received two weeks of pre-treatment and 12 weeks of post-treatment with the composition vs. bland moisturizer at POD 1-3 (FIG. 9A), POD 5-7 (FIG. 9B), and POD 10-14 (FIG. 9B). The images demonstrate reduced skin discoloration, ecchymosis, edema with the application of the composition before and after the procedure as compared to the control group with a bland moisturizer.

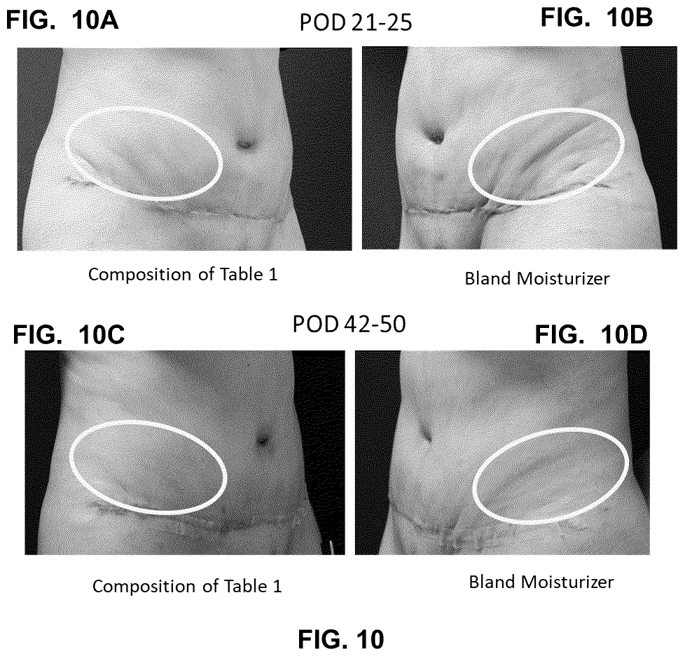

FIGS. 10A-D show images of the abdomen of a 38 year old subject after an abdominoplasty and liposuction who received two weeks of pre-treatment and 12 weeks of post-treatment with the composition vs. bland moisturizer at POD 21-25 (FIGS. 10A, B) and POD 42-50 (FIGS. 10C, D). The images demonstrate reduced fibrous banding in the areas indicated by a circle in FIGS. 10A and 10C the application of the composition before and after the procedure as compared to the control group with a bland moisturizer in FIGS. 10B and 10D.

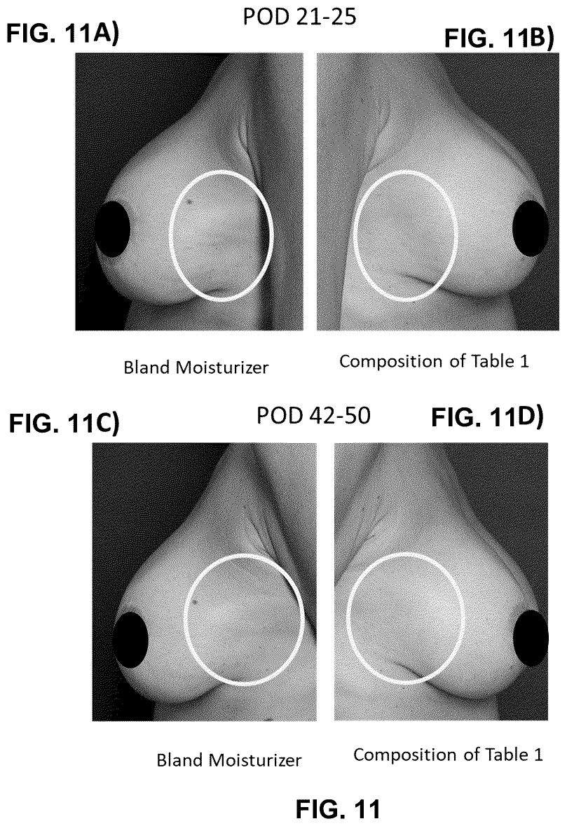

FIGS. 11A-D show images of the breast of a subject after a secondary breast augmentation who received two weeks of pre-treatment and 12 weeks of post-treatment with the composition vs. bland moisturizer at POD 21-25 (FIGS. 11B, A) and POD 42-50 (FIGS. 10D, C). The images demonstrate reduced swelling in the areas indicated by a circle in FIGS. 11B and 11D the application of the composition before and after the procedure as compared to the control group with a bland moisturizer in FIGS. 11A and 11C. The subject reported less swelling, less numb sensation, softer and more flexible skin with the composition treatment. Investigator assessment reported decreased fibrous banding on the treated side.

This double-blind, randomized, split-body study showed statistically significant improvements with treatment with the composition of Table 1 in at least ecchymosis, skin discoloration, edema, and subcutaneous fibrous banding. These data support that the composition may result in favorable outcomes during postsurgical healing following cosmetic surgical procedures. These data support gene expression studies in patients undergoing invasive body contouring surgery, where improvements in signs and symptoms started at 2 weeks after surgery (bruising/ecchymoses) when inflammation abated and were maximized at 4 weeks after surgery (skin discoloration, edema, fibrous banding) when regeneration began on gene expression.

### EMBODIMENTS

Numbered embodiment 1 comprises topical composition comprising: a tripeptide-1; a tetrapeptide-2; a hexapeptide-12; a hexapeptide-11; and a glycoprotein. Numbered embodiment 2 comprises the topical composition of numbered embodiment 1, wherein the tripeptide-1 is present in a range of about 0.05% by weight (wt. %) to about 5.00% wt. %. Numbered embodiment 3 comprises the topical composition of numbered embodiment 1, wherein the tripeptide-1 is present at about 3.00 wt. %. Numbered embodiment 4 comprises the topical composition of any one of numbered embodiments 1-3, wherein the tripeptide-1 comprises palmitoyl tripeptide-1, myristoyl tripeptide-1, or a combination thereof. Numbered embodiment 5 comprises the topical composition of any one of numbered embodiments 1-4, wherein the hexapeptide-12 comprises palmitoyl hexapeptide-12, myristoyl hexapeptide-12, or a combination thereof. Numbered embodiment 6 comprises the topical composition of any one of numbered embodiments 1-4, wherein the hexapeptide-12 is present in a range of about 0.05 wt. % to about 5 wt. %. Numbered embodiment 7 comprises the topical composition of any one of numbered embodiments 1-6, wherein the hexapeptide-12 is present at about 3.00 wt. %. Numbered embodiment 8 comprises the topical composition of any one of numbered embodiments 1-7, wherein the hexapeptide-11 is present in a range of about 0.002 wt. % to about 0.5 wt. %. Numbered embodiment 9 comprises the topical composition of any one of numbered embodiments 1-8, wherein the hexapeptide-11 is present at about 0.01 wt. %. Numbered embodiment 10 comprises the topical composition of any one of numbered embodiments 1-9, wherein the glycoprotein is a transferrin. Numbered embodiment 11 comprises the topical composition of any one of numbered embodiments 1-9, wherein the glycoprotein is a lactoferrin. Numbered embodiment 12 comprises the topical composition of numbered embodiment 11, wherein the lactoferrin is present in a range of about 0.01 wt. % to about 0.3 wt. %. Numbered embodiment 13 comprises the topical composition of numbered embodiment 11, wherein the lactoferrin is present at no more than about 0.01 wt. %. Numbered embodiment 14 comprises the topical composition of numbered embodiment 11, wherein the lactoferrin is present at about 0.05 wt. %. Numbered embodiment 15 comprises the topical composition of any one of numbered embodiments 1-14, further comprising phosphatidylserine. Numbered embodiment 16 comprises the topical composition of numbered embodiment 15, wherein the phosphatidylserine is present at no more than about 0.002 wt. %. Numbered embodiment 17 comprises the topical composition of numbered embodiment 15, wherein the phosphatidylserine is present in a range of about 0.002 wt. % to about 0.5 wt. %. Numbered embodiment 18 comprises the topical composition of numbered embodiment 15, wherein the phosphatidylserine is present at no more than about 5.0 wt. %. Numbered embodiment 19 comprises the topical composition of numbered embodiment 15, wherein the phosphatidylserine is present at about 0.1 wt. %. Numbered embodiment 20 comprises the topical composition of any one of numbered embodiments 1-19, further comprising oleuropein. Numbered embodiment 21 comprises the topical composition of numbered embodiment 20, wherein the oleuropein is present at no more than about 0.01 wt. %. Numbered embodiment 22 comprises the topical composition of numbered embodiment 20, wherein the oleuropein is present in a range between about 0.01 wt. % and about 0.30 wt. %. Numbered embodiment 23 comprises the topical composition of numbered embodiment 20, wherein the oleuropein is present at about 0.05 wt. %. Numbered embodiment 24 comprises the topical composition of any one of numbered embodiments 1-23, further comprising Tremella fuciformis extract. Numbered embodiment 25 comprises the topical composition of numbered embodiment 24, wherein the Tremella fuciformis extract is present at no more than about 0.10 wt. %. Numbered embodiment 26 comprises the topical composition of numbered embodiment 24, wherein the Tremella fuciformis extract is present in a range of about 0.10 wt. % to about 3.00 wt. %. Numbered embodiment 27 comprises the topical composition of numbered embodiment 24, wherein the Tremella fuciformis extract is present at about 0.50 wt. %. Numbered embodiment 28 comprises the topical composition of any one of numbered embodiments 1-27, further comprising Peucedanum graveolens extract. Numbered embodiment 29. The topical composition of numbered embodiment 28, wherein the Peucedanum graveolens extract is present at no more than about 0.10 wt. %. Numbered embodiment 30 comprises the topical composition of numbered embodiment 28, wherein the Peucedanum graveolens extract is present in a range of about 0.10 wt. % to about 3.00 wt. %. Numbered embodiment 31 comprises the topical composition of numbered embodiment 28, wherein the Peucedanum Numbered embodiment extract is present at about 0.50 wt. %. Numbered embodiment 32 comprises the topical composition of any one of numbered embodiments 1-31, further comprising hydroxymethoxyphenyl decanone. Numbered embodiment 33 comprises the topical composition of numbered embodiment 32, wherein the hydroxymethoxyphenyl decanone is present at no more than about 0.10 wt. %. Numbered embodiment 34 comprises the topical composition of numbered embodiment 32, wherein the hydroxymethoxyphenyl decanone is present in a range of about 0.10 wt. % to about 3.00 wt. %. Numbered embodiment 35 comprises the topical composition of numbered embodiment 32, wherein the hydroxymethoxyphenyl decanone is present at about 0.50 wt. %. Numbered embodiment 36 comprises the topical composition of any one of numbered embodiments 1-35, further comprising Dunaliella salina extract. Numbered embodiment 37 comprises the topical composition of numbered embodiment 36, wherein the Dunaliella salina extract is present at no more than about 0.10 wt. %. Numbered embodiment 38 comprises the topical composition of numbered embodiment 36, wherein the Dunaliella salina extract is present in a range of about 0.10 wt. % to about 3.00 wt. %. Numbered embodiment 39 comprises the topical composition of numbered embodiment 36, wherein the Dunaliella salina extract is present at about 0.50 wt. %. Numbered embodiment 40 comprises the topical composition of any one of numbered embodiments 1-39, further comprising Ledum palustre. Numbered embodiment 41 comprises the topical composition of numbered embodiment 40, wherein the Ledum palustre is present at no more than about 0.10 wt. %. Numbered embodiment 42 comprises the topical composition of numbered embodiment 40, wherein the Ledum palustre is present in a range of about 0.10 wt. % to about 3.00 wt. %. Numbered embodiment 43 comprises the topical composition of numbered embodiment 40, wherein the Ledum palustre is present at about 0.50 wt. %. Numbered embodiment 44 comprises the topical composition of any one of numbered embodiments 1-43, further comprising xylitylglucoside. Numbered embodiment 45 comprises the topical composition of numbered embodiment 44, wherein the xylitylglucoside is present at no more than about 0.2 wt. %. Numbered embodiment 46 comprises the topical composition of numbered embodiment 44, wherein the xylitylglucoside is present in a range of about 0.2 wt. % to about 5.00 wt. %. Numbered embodiment 47 comprises the topical composition of numbered embodiment 44, wherein the xylitylglucoside is present at about 1.00 wt. %. Numbered embodiment 48 comprises the topical composition of any one of numbered embodiments 1-47, further comprising anhydroxylitol. Numbered embodiment 49 comprises the topical composition of numbered embodiment 48, wherein the anhydroxylitol is present at no more than about 0.2 wt. %. Numbered embodiment 50 comprises the topical composition of numbered embodiment 48, wherein the anhydroxylitol is present in a range of about 0.2 wt. % to about 5.00 wt. %. Numbered embodiment 51 comprises the topical composition of numbered embodiment 48, wherein the anhydroxylitol is present at about 1.00 wt. %. Numbered embodiment 52 comprises the topical composition of any one of numbered embodiments 1-51, further comprising xylitol. Numbered embodiment 53 comprises the topical composition of numbered embodiment 52, wherein the xylitol is present at no more than about 0.2 wt. %. Numbered embodiment 54 comprises the topical composition of numbered embodiment 52, wherein the xylitol is present in a range of about 0.2 wt. % to about 5.00 wt. %. Numbered embodiment 55 comprises the topical composition of numbered embodiment 52, wherein the xylitol is present at about 1.00 wt. %. Numbered embodiment 56 comprises the topical composition of any one of numbered embodiments 1-43, further comprising xylitylglucoside, anhydroxylitol, and xylitol. Numbered embodiment 57 comprises the topical composition of numbered embodiment 56, wherein the xylitylglucoside, anhydroxylitol, and xylitol are present at no more than about 0.2 wt. %. Numbered embodiment 58 comprises the topical composition of numbered embodiment 56, wherein the xylitylglucoside, anhydroxylitol, and xylitol are present in a range of about 0.2 wt. % to about 5.00 wt. %. Numbered embodiment 59 comprises the topical composition of numbered embodiment 56, wherein the xylitylglucoside, anhydroxylitol, and xylitol are present at about 1.00 wt. %. Numbered embodiment 60 comprises the topical composition of any one of numbered embodiments 1-59, further comprising Centella asiatica extract. Numbered embodiment 61 comprises the topical composition of numbered embodiment 60, wherein the Centella asiatica extract is present at no more than about 0.2 wt. %. Numbered embodiment 62 comprises the topical composition of numbered embodiment 60, wherein the Centella asiatica extract is present in a range of about 0.2 wt. % to about 5.00 wt. %. Numbered embodiment 63 comprises the topical composition of numbered embodiment 60, wherein the Centella asiatica extract is present at about 1.00 wt. %. Numbered embodiment 64 comprises the topical composition of any one of numbered embodiments 1-63, further comprising naringenin. Numbered embodiment 65 comprises the topical composition of numbered embodiment 64, wherein the naringenin is present at no more than about 0.4 wt. %. Numbered embodiment 66 comprises the topical composition of numbered embodiment 64, wherein the naringenin is present in a range of about 0.4 wt. % to about 10.00 wt. %. Numbered embodiment 67 comprises the topical composition of numbered embodiment 64, wherein the naringenin is present at about 2.00 wt. %. Numbered embodiment 68 comprises the topical composition of any one of numbered embodiments 1-67, further comprising Arnica montana extract. Numbered embodiment 69 comprises the topical composition of numbered embodiment 68, wherein the Arnica montana extract is present at no more than about 0.10 wt. %. Numbered embodiment 70 comprises the topical composition of numbered embodiment 68, wherein the Arnica montana extract is present in a range of about 0.10 wt. % to about 3.00 wt. %. Numbered embodiment 71 comprises the topical composition of numbered embodiment 68, wherein the Arnica montana extract is present at about 0.50 wt. %. Numbered embodiment 72 comprises the topical composition of any one of numbered embodiments 1-71, further comprising tetrandrine. Numbered embodiment 73 comprises the topical composition of numbered embodiment 72, wherein the tetrandrine is present at no more than about 0.0002 wt. %. Numbered embodiment 74 comprises the topical composition of numbered embodiment 72, wherein the tetrandrine is present in a range of about 0.0002 wt. % to about 0.005 wt. %. Numbered embodiment 75 comprises the topical composition of numbered embodiment 72, wherein the tetrandrine is present at about 0.001 wt. %. Numbered embodiment 76 comprises the topical composition of any one of numbered embodiments 1-75, wherein the topical composition is aqueous.

Numbered embodiment 77 comprises a method for improving or preventing effects of a body contouring or surgical procedure comprising administering the topical composition of any one of numbered embodiments 1-76.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A topical composition comprising:
a tripeptide-1;
a tetrapeptide-2;
a hexapeptide-12;
a hexapeptide-11; and
a glycoprotein.

2. The topical composition of claim 1, wherein the tripeptide-1 is present at a concentration of about 0.05 wt. % to about 5.00% wt. %, relative to the total weight of the composition.

3. The topical composition of claim 1, wherein the tripeptide-1 comprises palmitoyl tripeptide-1, myristoyl tripeptide-1, or a combination thereof.

4. The topical composition of claim 1, wherein the hexapeptide-12 is present at a concentration of about 0.05 wt. % to about 5 wt. %, relative to the total weight of the composition.

5. The topical composition of claim 1, wherein the hexapeptide-12 comprises palmitoyl hexapeptide-12, myristoyl hexapeptide-12, or a combination thereof.

6. The topical composition of claim 1, wherein the hexapeptide-11 is present at a concentration of about 0.002 wt. % to about 0.5 wt. %, relative to the total weight of the composition.

7. The topical composition of claim 1, wherein the tetrapeptide-2 is present at a concentration of about 0.2 wt. % to about 5 wt. %, relative to the total weight of the composition.

8. The topical composition of claim 1, wherein the glycoprotein comprises lactoferrin.

9. The topical composition of claim 1, wherein the glycoprotein is present at a concentration of 0.001 wt. % to about 6 wt. %, relative to the total weight of the composition.

10. The topical composition of claim 1, wherein the composition further comprises one or more of phosphatidylserine, oleuropein, Tremella fuciformis extract, Peucedanum graveolens extract, hydroxymethoxyphenyl decanone, Dunaliella salina extract, Ledum palustre, xylitylglucoside, anhydroxylitol, xylitol, Centella asiatica extract, naringenin, Arnica montana extract, or tetrandrine.

11. The topical composition of claim 1, wherein the composition enhances TFG-β3 expression.

12. A method of preventing or improving effects associated with a body contouring or surgical procedure, the method comprising:
administering a composition according to claim 1 to skin of a subject.

13. The method of claim 12, wherein the composition is administered before the body contouring or surgical procedure.

14. The method of claim 12, wherein the composition is administered after the body contouring or surgical procedure.

15. The method of claim 12, wherein the body contouring or surgical procedure comprises a non-invasive procedure.

16. The method of claim 12, wherein the body contouring or surgical procedure comprises an invasive procedure.

17. The method of claim 12, wherein the body contouring or surgical procedure comprises abdominoplasty, liposuction, bariatric surgery, breast reduction, breast augmentation, breast lift, breast reconstruction, thigh lift, fat grafting, fat reduction, forehead lift, cheek enhancement, otoplasty, a reconstructive procedure, rhytidectomy, lower rhytidectomy, cheek reduction, mentoplasty, blepharoplasty, facial implant, nose surgery, skin excision, skin biopsy, invasive cellulite treatment, an injection, microneedling, a procedure to reduce visibility of veins, or combinations thereof.

18. The method of claim 12, wherein the body contouring or surgical procedure comprises low level laser therapy, infrared light, high frequency focused ultrasound, pulsed focus ultrasound, radiofrequency, radiofrequency induced electroporation, injectable lipolytic agents, cryolipolysis, or a combination thereof.

19. The method of claim 12, wherein the effects associated with a body contouring or surgical procedure comprise bruising, swelling, fat dissolution, antimicrobial effects, skin tightening, scarring, or any combination thereof.

20. The method of claim 12, wherein the composition modulates expression of TGF-β1, TGF-β3, IL-10, IL-6, SMAD-2, SMAD-3, SMAD-4, SMAD-7, COX-2, IL-β, TNF-α, tissue inhibitors of metalloproteinase (TIMP), MMP-1, VEGF, or combinations thereof.
